(19) United States
(12) Patent Application Publication
(10) Pub. No.: US 2026/0182836 A1
(43) Pub. Date: Jul. 2, 2026

(54) OPHTHALMIC CONTACT LENS WITH INTEGRATED OR ATTACHABLE ILLUMINATION AND IMAGING CAPABILITIES

(71) Applicant: EyeX Solutions Inc., San Juan Capistrano, CA (US)

(72) Inventor: Christopher HORVATH, San Juan Capistrano, CA (US)

(21) Appl. No.: 19/417,024

(22) Filed: Dec. 11, 2025

Related U.S. Application Data
(60) Provisional application No. 63/730,930, filed on Dec. 11, 2024.
(60) Provisional application No. 63/812,867, filed on May 28, 2025.

(51) Int. Cl. A61B 3/117; A61B 3/15

(52) U.S. Cl. CPC A61B 3/117; A61B 3/152

(57) ABSTRACT

Systems and methods for illuminating and imaging an eye during ophthalmic procedures are disclosed. An ophthalmic apparatus includes an ophthalmic lens body configured for placement on or near a patient's eye and at least one miniature illumination source coupled to the lens body. The illumination source is positioned to emit light towards an intraocular structure and moves in unison with the lens body, thereby maintaining consistent, automatically aligned illumination of the target region without requiring external light sources or manual readjustment. Optionally, the apparatus can include illumination sources integrated directly into the lens or provided as a detachable, self-powered ring or cap. The apparatus may further incorporate miniature cameras for imaging and guidance, specialized illumination modalities such as selectable wavelengths or polarization, and alignment apertures to ensure safe and accurate delivery of therapeutic laser beams. The systems can enhance visualization, improve diagnostic consistency, and streamline surgical workflows.

### CROSS-REFERENCE TO RELATED APPLICATIONS

This application claims the benefit of and priority to U.S. Provisional Application No. 63/730,930, filed on Dec. 11, 2024, and U.S. Provisional Application No. 63/812,867, filed on May 28, 2025, the entire contents of which is incorporated herein by reference.

### BACKGROUND

### Field of the Inventions

The present inventions generally relate to ophthalmic devices and techniques for illuminating and visualizing the interior of the eye during diagnostic procedures and eye surgeries. More particularly, it pertains to improved contact lens devices and illumination systems that provide optimized, targeted lighting to facilitate diagnostic imaging, laser treatments, and surgical interventions in the eye.

### Description of the Related Art

During ophthalmic examinations and surgical procedures, illumination is typically provided by external sources such as a slit lamp apparatus, a surgical microscope's built-in light, a head-mounted lamp, or a handheld probe. For example, in laser treatments like selective laser trabeculoplasty (SLT), argon laser trabeculoplasty (ALT), or YAG capsulotomy, a slit lamp delivers a light beam alongside the microscope optics. The clinician must adjust parameters like slit width, beam angle, intensity, and orientation to illuminate the target tissue appropriately. When using a gonioscopic lens (a contact lens with mirror(s) for viewing the eye's anterior angle) or any other lens (with no mirrors or multiple mirrors) in conjunction with such external illumination, the surgeon typically holds the lens on the patient's eye with one hand and operates the slit lamp or microscope with the other thereby switching one hand between illumination adjustments and slit lamp steering operation.

### SUMMARY

In accordance with at least some embodiments disclosed herein is the realization that continuous fine-tuning is often needed to maintain optimal lighting as the viewing angle changes. In accordance with at least some embodiments disclosed herein is the realization that misalignment or suboptimal adjustment can cause uneven illumination, shadows, or glare. For example, in some cases, excessive light accidentally directed through the pupil can reach the retina, causing patient discomfort or transient flash blindness.

In accordance with at least some embodiments disclosed herein is the realization that adjustable built-in illumination from slit lamps or fixed light sources in surgical microscopes do not automatically follow the angle of view of a gonio or diagnostic/treatment lens. There is often a mismatch between the fixed/adjustable illumination angle and the field of view provided by an angled mirror lens during gonioscopy or laser treatment. As a result, important structures (e.g., the trabecular meshwork in the anterior chamber angle) may not be evenly lit when viewed through the gonio lens mirror, requiring repeated adjustments of the external light source. Moreover, in systems where a surgical microscope and a therapeutic laser share the optical path (dual-path systems), the beam-splitting optics can complicate illumination. The built-in light may be partially obstructed or spectrally or generally attenuated by intervening mirrors/prisms, making it hard to achieve bright, uniform illumination of the target. Surgeons often lack the ability to easily change the color or spectrum of the illumination in such systems, whereas certain anatomical details (like pigmented trabecular meshwork vs. surrounding tissue) might be seen more clearly under specific wavelengths. These optical challenges highlight the need for more flexible illumination solutions (for example, the ability to use customized color LEDs near the target region) in order to enhance contrast and visibility of ocular structures.

Surgeon Workload and Precision Demands: In accordance with at least some embodiments disclosed herein is the realization that because current illumination systems are not automatically aligned with the internal views, the surgeon must constantly divide attention between holding the contact lens on the eye, operating foot pedals or controls for the laser, and manually tweaking the light beam's position and settings. This multitasking can prolong procedure time and increase the risk of suboptimal treatment. The problem is exacerbated in high-precision laser surgeries, such as those using femtosecond or picosecond lasers, which demand extremely accurate focal plane positioning. In such cases, consistent, high-quality illumination is critical for the surgeon to confidently align the laser focus plane with the target tissue. Any fluctuation or unevenness in lighting can make it difficult to judge depth and focus, potentially affecting treatment accuracy.

Risks with Emerging Laser Technologies: In accordance with at least some embodiments disclosed herein is the realization that modern ophthalmic lasers with large beam diameters and high numerical apertures introduce additional challenges. For example, certain photodisruptive lasers focus to a very small spot (a few micrometers) at the target, but their entry beam at the contact lens can be several millimeters wide. If the contact lens is even slightly misaligned relative to the laser beam, part of the laser can be clipped by the lens edge or any attached structures, reducing laser energy delivery and altering the spot quality. Without a dedicated alignment aid, the surgeon must rely on guesswork or indirect cues to detect such misalignment. Fixed or adjustable external illumination offers no help in this regard and may even contribute to glare that hides the subtle signs of beam clipping. There is a clear need for an illumination system that inherently aligns with the therapeutic beam path and provides visual feedback (for instance, through an alignment aperture or indicator) to ensure the laser beam is unobstructed.

Diagnostic Inconsistencies: In accordance with at least some embodiments disclosed herein is the realization that in diagnostic gonioscopy and anterior chamber evaluation, different examiners or sessions might use different illumination settings, making it difficult to consistently assess findings like trabecular meshwork (TM) pigmentation or to identify landmarks such as Schwalbe's line. Variable lighting can cause certain features to be missed or appear differently. A standardized, reproducible illumination condition would improve the reliability of diagnosing conditions (for example, grading of angle pigmentation or detecting narrow angles) by ensuring the eye's structures are viewed under the same lighting each time.

Reusable vs. Disposable Instruments: Most ophthalmic contact lenses (gonio lenses, three-mirror lenses, etc.) are reusable and must be sterilized between patients. In accordance with at least some embodiments disclosed herein is the realization that any illumination attachments (fiber-optic cables, light guides, etc.) can further complicate handling and may tether the lens, restricting the surgeon's maneuverability. There is an infection control concern whenever equipment is reused or comes into contact with the ocular surface, especially in delicate intraocular procedures. A self-contained, battery-powered illumination device that is single-use (disposable) could eliminate the need for bulky external light cables and remove the risk of cross-contamination, while also giving the surgeon full freedom to rotate or position the lens as needed without fighting attached cords.

In summary, at least some embodiments relate to the realization that conventional approaches to ophthalmic illumination are labor-intensive and limited in their ability to provide optimal, angle-independent lighting. They require constant manual intervention, may cause patient discomfort, and are not easily adaptable to new high-precision laser techniques or advanced imaging guidance. These shortcomings underscore the need for improved systems and methods that deliver pre-aligned, high-quality illumination directly at the site of interest, thereby reducing surgeon workload and improving visualization and treatment accuracy.

The present disclosure addresses the above challenges by integrating one or more miniature illumination sources directly with ophthalmic contact lens or patient interface devices used during eye examinations and surgeries. In contrast to relying on an external slit lamp or microscope light, some embodiments of the devices disclosed herein provide built-in illumination at the level of the contact lens (for example, in a gonioscopic lens or a diagnostic contact lens). Because the light travels with the lens, it remains automatically aligned with the viewing optics and target tissue as the lens is rotated or moved, thereby maintaining consistent illumination on the area of interest without continual readjustment.

In accordance with some embodiments, a gonioscopic lens assembly (having one or more mirrors for angle viewing) is equipped with integrated light-emitting diodes (LEDs) or other miniature light sources mounted or embedded near the mirror surface. These light sources are fixed in position and directed relative to the mirror, so that they always illuminate the region of the eye that the mirror is viewing. For example, when the gonio lens mirror is directed at the trabecular meshwork in the anterior chamber angle, the embedded LED shines directly into the angle, following the same path the surgeon is observing or the laser will follow. This approach ensures that optimal lighting is delivered precisely where needed (e.g., the laser target or diagnostic region), independent of any external lamp. By providing illumination at the point of care, some embodiments can be configured to simplify the surgical workflow: once the device is in place and its internal light is activated, the surgeon can proceed with treatment or examination without pausing to reposition external illumination.

In another aspect, some embodiments can be configured to include illumination ring or cap attachments that mount on the contact lens device. For instance, a removable ring-shaped illumination module can be placed around the top of a gonioscopic lens or a three-mirror lens. This ring carries multiple small LED lights arranged around a central aperture. The central opening aligns with the main optical pathway (the viewing port or laser path through the lens), ensuring that the surgeon's view and any treatment beam pass unobstructed through the middle of the ring. The LEDs on the ring uniformly illuminate the periphery and target tissue without blocking the line of sight. Such a ring can be designed as a single-use disposable cap that snaps onto the lens before use and is removed afterwards, containing its own power source (e.g., a micro-battery) and, optionally, control electronics. This configuration not only guarantees a fresh, sterile light source for each procedure (eliminating sterilization concerns) but also avoids any cumbersome fiber optic cables, since the ring is self-powered and wire-free.

The illumination provided by some embodiments can be tailored in spectrum and intensity to enhance visualization of specific tissues. LEDs of different colors (white, red, green, blue, infrared, etc.) may be used alone or in combination to highlight certain anatomical details. For example, a green or blue light might improve contrast of blood vessels or pigmented structures, while infrared illumination could be used in conjunction with an IR-sensitive camera to view structures behind the iris or within the trabecular meshwork that are not visible under visible light. The integrated nature of the illumination means these specialized lighting modes can be easily activated without changing external equipment—potentially by a switch on the lens assembly or a remote control—thus expanding the diagnostic capabilities in real-time.

Optionally, some embodiments can be configured to comprise miniature cameras or sensors into the contact lens assembly alongside the illumination sources. A tiny camera (on the order of 1-2 mm) embedded in or attached to the lens could capture live images or video of the anterior chamber or retina during a procedure. Paired with the built-in lights, this allows for automated guidance or documentation: for example, a computer system could analyze the real-time image to assist in aligning a laser (auto-guidance) or to record the procedure for later review. Because the camera shares the same field of view and illumination, the imagery remains consistent and well-lit. In some embodiments, multiple such cameras or optical sensors are distributed around the lens (or ring) to give a stereoscopic or wide-field view inside the eye. All of these components—lights and cameras—can be incorporated without significantly increasing the size of the lens apparatus, given modern microelectronics, thereby preserving the ergonomics for the surgeon.

In summary, some embodiments can provide systems and methods for illuminating and imaging the eye that are built into the contact lens instruments themselves. By moving the light source from an external system to the lens on the eye, some embodiments can achieve precise, stable, and shadow-free illumination of the target tissue with minimal user adjustment. This results in improved visualization (the surgeon sees the anatomy clearly, with less glare and more uniform light), enhanced diagnostic capability (consistent lighting for comparing clinical observations and the option of integrated imaging), and greater treatment accuracy (particularly for high-precision lasers that require exact alignment and focus). The surgeon's workload is reduced because the system is effectively self-aligning: once the lens with its illumination is in place, the illumination automatically tracks any movement of the lens. The end result is a significant advancement over conventional techniques, providing a safer, more efficient, and higher-fidelity approach to ophthalmic illumination and imaging.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is a cross-sectional side view of a gonioscopic lens positioned on an eye, illustrating an exemplary beam path.

FIG. 2 is a top-down view of the gonioscopic lens of FIG. 1, showing integrated illumination sources positioned near the internal mirror.

FIG. 3 is a cross-sectional view taken along line A-A of FIG. 2, illustrating the orientation of the integrated illumination sources.

FIG. 4 is a cross-sectional view illustrating a conventional external illumination scenario and the potential for stray light reaching the retina.

FIG. 5 is a cross-sectional view illustrating a beam path during a tilted gonioscopic procedure.

FIG. 6 is an exploded side view showing a detachable illumination cap relative to a gonioscopic lens and an eye.

FIG. 7 is a perspective view of the assembled illumination cap mounted on the gonioscopic lens and positioned on the eye.

FIG. 8 is a side elevational view of the assembly shown in FIG. 7.

FIG. 9 is a top view of an illumination cap embodiment illustrating integrated components including light sources, a battery, sensors, and an alignment aperture.

FIG. 10 is a top plan view of an illumination system indicating cross-section line B-B.

FIG. 11 is a cross-sectional view taken along line B-B of FIG. 10, illustrating simultaneous angle illumination and central illumination for pupil modulation.

FIG. 12 is a cross-sectional view illustrating the detection of laser beam misalignment and clipping using an alignment aperture.

FIG. 13 is a front view assembly diagram illustrating the alignment of an illumination ring, gonioscopic lens, and eye.

FIG. 14 is a cross-sectional view of a lens assembly combining multiple illumination modalities, including a top-mounted cap and embedded sidewall lights.

FIG. 15(a)-(d) are side views of various ophthalmic contact lenses.

FIG. 16 is a perspective view of a partially open illumination cap mounted on a gonioscopic lens on an eye.

FIG. 17 is a top view of the partially open illumination cap shown in FIG. 16.

FIG. 18 is a top view of another illumination ring embodiment featuring a large central opening.

FIG. 19(a)-(b) are cross-sectional views illustrating wide-angle retina lenses.

FIG. 20 is a top view of an illumination ring adapted for vitreoretinal applications, featuring multiple illumination and imaging units.

FIG. 21 is a cross-sectional side view of a direct contact lens with integrated peripheral and central illumination modules.

FIG. 22(a)-(b) are perspective views of an illumination ring with integrated LEDs, stereoscopic cameras, and a battery, mounted on a gonioscopic lens.

FIG. 23(a)-(b) are a side elevational view and an exploded perspective view of the assembly of FIG. 22, highlighting angled LED channels.

FIG. 24(a)-(b) are a top view and a perspective view of a compact illumination ring variant featuring a horizontally oriented battery.

FIG. 25 is an exploded view of the compact ring assembly of FIG. 24, illustrating optional external power and data connectivity.

FIG. 26 is an exploded view of an illumination ring assembly adapted for a three-mirror diagnostic contact lens.

FIG. 27(a)-(c) are a top view, a cross-sectional view (SECTION A-A), and a detailed view (B) of the assembled three-mirror lens illumination system of FIG. 26.

FIG. 28(a)-(b) are a side cross-sectional view and a top view of an illumination ring mounted on a double-mirror gonioscopic lens.

FIG. 29(a)-(b) are an exploded view and a top view of an illumination module embodiment featuring integrated control electronics.

FIG. 30(a)-(b) are a top view and a cross-sectional view (B-B) of an illumination module illustrating angled mounting of LEDs or cameras with optional focusing optics.

FIG. 31 is a block diagram illustrating various configurations of the illumination system components, power sources, and control signals.

### DETAILED DESCRIPTION

The following detailed description presents various embodiments, with reference to the accompanying drawings. Throughout, like reference numerals indicate similar or corresponding features in the figures. It is to be understood that the described embodiments are examples provided to enable understanding of the present disclosure, and that numerous variations and modifications can be made without departing from the scope of the inventions, as defined by the claims

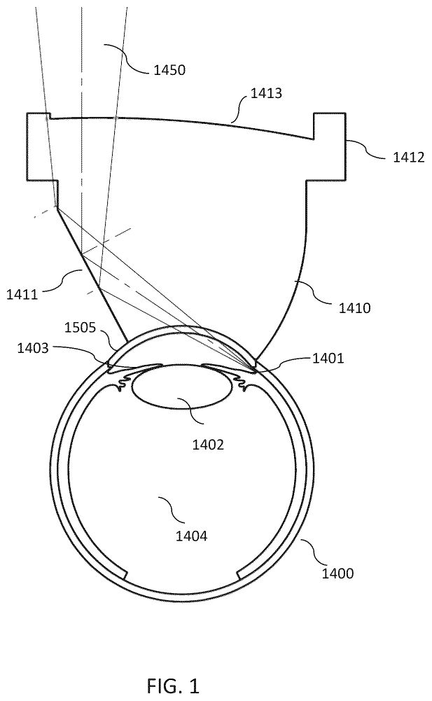

FIG. 1 is a cross-sectional side view of a one-mirror gonioscopic lens 1410 in use on a patient's eye 1400. The lens 1410 has an upper gripping ring 1412 for handling and a curved top optical surface 1413 (which may act as a magnifying lens). An internal mirror 1411 is positioned on one side of the gonio lens. In this view, the gonio lens 1410 is placed on the cornea 1405 of the eye and interfaces with the anterior chamber angle region 1401. Also illustrated schematically are anatomical elements of the eye: the crystalline lens 1402, the iris 1403, the vitreous body 1404, and the general region of the trabecular meshwork and Schlemm's canal (within 1401). An exemplary imaging or focused laser beam path 1450 is shown entering from the left side, passing through the lens 1410, reflecting off the mirror 1411, and focusing into the anterior angle region 1401.

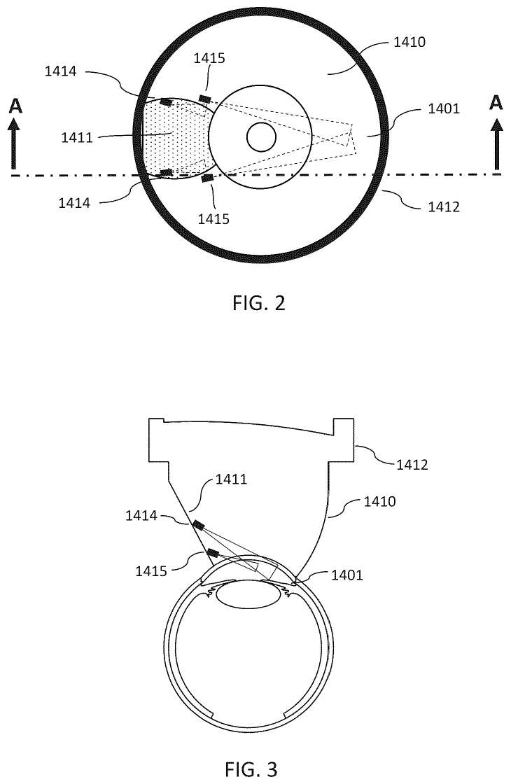

FIG. 2 is a top-down plan view of the gonioscopic lens 1410 (as in FIG. 1), illustrating one embodiment of integrated illumination. In this figure, the gonio lens 1410 has a single mirror 1411 on the left side (the shaded semi-circular area). Four miniature light sources 1414, 1415 are embedded in the lens body around the mirror 1411. Two light sources 1414 are placed at the left edge of the mirror surface 1411, within the footprint of the mirror but towards its periphery, so as not to obstruct the central reflective area. Two additional light sources 1415 are positioned near the top and bottom of the lens (in this example) around the mirror region. The dashed lines emanating from the LEDs indicate their light cones directed toward the target area in the anterior angle. The arrows “A-A” indicate the cross-section plane for FIG. 3.

FIG. 3 is a cross-sectional view taken along line A-A of FIG. 2, showing the gonio lens 1410 with integrated LEDs in profile. This view illustrates how the light sources 1414, 1415 are oriented. The two LEDs 1414 near the mirror are tilted inward (toward the lens's central axis) and downward, aiming their light toward the eye's anterior angle region 1401 that the mirror 1411 reflects. The other two LEDs 1415 are similarly directed toward the same target region. Because the gonio lens 1410 is in contact with the eye 1400 (cornea 1405), the emitted light enters the anterior chamber without significant loss. As the lens is rotated by its ring handle 1412 to view different portions of the angle, the LEDs 1414, 1415 move with it, continuously lighting the region in the mirror's field of view.

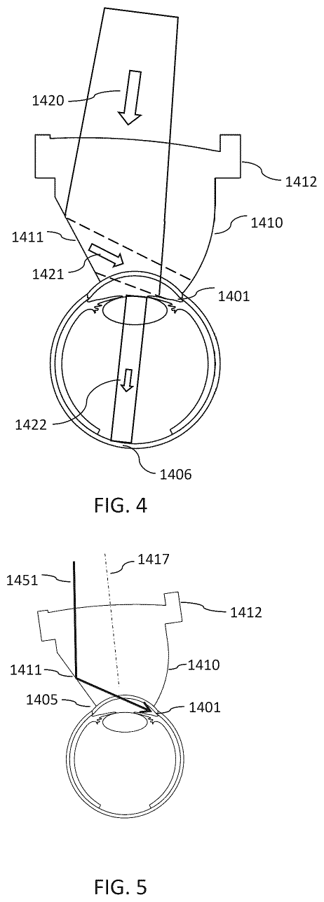

FIG. 4 is a cross-sectional illustration of a conventional illumination scenario using a gonioscopic lens, in accordance with conventional methodologies. A broad illumination beam 1420 (for example, from a slit lamp) is directed toward the gonio lens 1410. Part of this beam (labeled 1421) reflects off the mirror 1411 toward the intended target in the angle 1401, but another portion 1422 passes through the pupil of the iris and reaches the retina 1406 at the back of the eye. Because the external beam 1420 is relatively large and unfocused, it illuminates areas beyond the target, potentially causing glare or discomfort. FIG. 4 thus demonstrates the drawback of external illumination: even with proper aim, some light can bypass the target (trabecular meshwork) and shine into the eye's interior (arrow toward 1406), which may distract or temporarily blind the patient.

FIG. 5 is a cross-sectional view of a somewhat tilted gonioscopic procedure. In this figure, a diagnostic or laser beam 1451 is shown entering the gonio lens 1410 and reflecting from mirror 1411 to the anterior angle 1401. Item 1417 illustrates the central axis of the gonio lens. The tilt angle between the incoming beam 1451 and the central axis 1417 illustrates how a goniolens or other mirrored lens may need to be tilted to enable correct targeting.

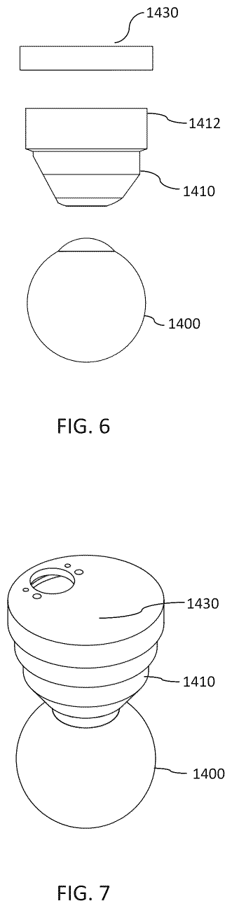

FIG. 6 is an exploded side view showing a disposable illumination ring or cap 1430 in relation to a gonioscopic lens 1410 and a patient's eye 1400. The ring/cap 1430 is depicted separated above the gonio lens 1410, illustrating that it is a distinct component designed to attach to the top of the lens. The eye 1400 is below the lens. This view demonstrates how the ring 1430 can be a modular piece that is added onto the lens assembly before use.

FIG. 7 is a perspective view of the illumination cap 1430 mounted on the gonioscopic lens 1410, which is in turn placed on the eye 1400. In FIG. 7, the assembled device is shown from an angled viewpoint. The illumination cap 1430 covers the upper portion of the lens and has a central opening. One or more apertures or recesses can be seen on the cap (for example, an alignment hole 1433 and possibly openings for lights or sensors), while the lower part of the gonio lens 1410 (with its tiered structure for mirrors) sits on the eye 1400. This figure helps visualize the overall form factor of the combined lens and light cap in use.

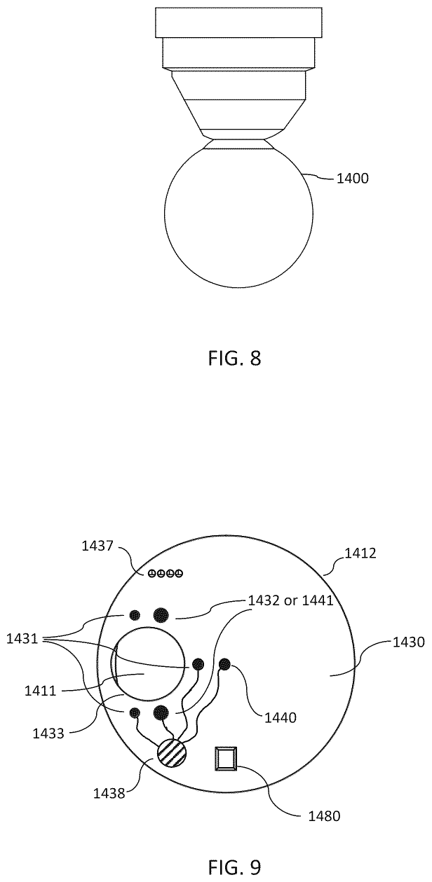

FIG. 8 is a side elevational view of the gonioscopic lens with the illumination cap from FIG. 7.

FIG. 9 is a top view of a gonio lens assembly with an attached illumination cap 1430, illustrating various integrated components. In this embodiment, the cap 1430 encircles the lens's upper rim 1412, leaving a central alignment aperture 1433 over the mirror area (the open circular area in the center). Multiple illumination elements 1431, 1432 are arranged around the perimeter of the aperture on the cap. For example, two illumination elements 1431 are positioned on one side and two elements 1432 on the opposite side, providing a ring of lights. A central element 1440 is shown at approximately the middle of the cap—this could be an optional camera or additional down-firing light that points through the central opening. A small battery 1438 is depicted on the cap (for instance, at the bottom area in the figure) to power the lights and any electronics. Additionally, a set of status indicator lights 1437 (drawn as a cluster of small circles near the top of the cap) is included to convey information (such as power on, battery charge level, laser ready, etc.). FIG. 9 thus provides a comprehensive view of a light cap embodiment, with labeled features including the main lens mirror 1411 (beneath the cap), the alignment hole 1433, the ring lights 1431/1432, the central module 1440, the battery 1438, and indicator LEDs 1437.

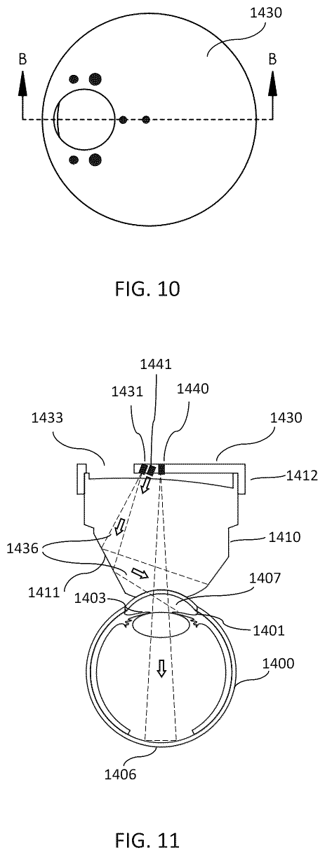

FIG. 10 is a plan view of another embodiment of the illumination system, shown from above. In FIG. 10, a contact lens (such as a gonio lens 1410 under a cap 1430) has an arrangement of elements including peripheral lights 1431 and a central aperture 1433 (similar to FIG. 9). The figure also indicates a cross-sectional cut line B-B across the device. This view may represent a configuration where certain parts of the illumination ring are open (as suggested by an asymmetric layout of components). It sets the stage for the cross-section in FIG. 11.

FIG. 11 is a cross-sectional side view taken along line B-B of FIG. 10, depicting the gonioscopic lens 1410 with an open-style illumination cap 1430 and a central down-looking element 1440. In this figure, the gonio lens 1410 is on the eye 1400 as before, and the cap 1430 is attached on top. The cap in this embodiment has a large open region over the right side (labeled 1439 in related figures) allowing a direct view of the pupil and iris. A central illumination/camera module 1440 is positioned over the center of the lens, emitting a beam (or viewing) straight down through the patient's pupil onto the retina 1406. Two illumination elements 1431 are shown on the left side of the cap, directing light toward the mirror 1411 and angle region 1401 as usual. The iris 1403 is shown in two positions: its normal state and a constricted state 1407 (for instance, after being stimulated by the central light 1440). When the central light 1440 shines into the eye and onto the retina, it causes a pupillary light reflex, making the iris contract (1407). As the iris constricts, its peripheral edge at the angle becomes thinner and pulls away from the trabecular meshwork area, thereby opening up the angle view. FIG. 11 illustrates how using a central illumination (1440) to deliberately constrict the pupil can improve access to the anterior angle by reducing iris interference. An alignment aperture 1433 is also visible in this cross-section, and an example of a light ray path 1436 from one of the ring LEDs toward the angle is indicated. Overall, FIG. 11 demonstrates the multi-functional use of the integrated system: simultaneous angle illumination and a central light to modulate the pupil or provide through-pupil visualization.

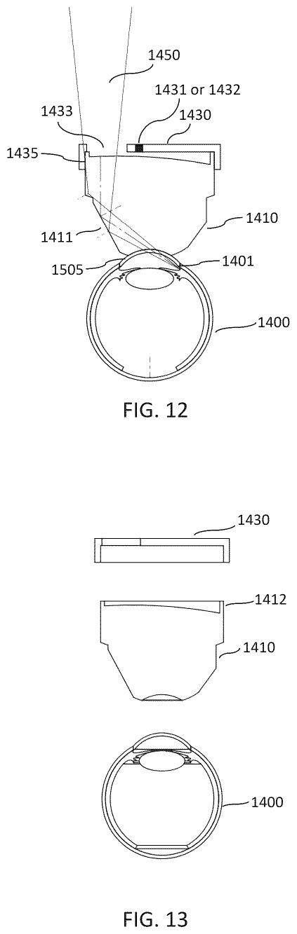

FIG. 12 is a cross-sectional diagram illustrating the issue of laser beam misalignment and the use of an alignment aperture. In FIG. 12, a gonio lens 1410 with an illumination ring 1430 is on the eye 1400 (similar to earlier figures). A high-aperture laser beam 1450 is shown entering from above but shifted off-center toward the left. The gonio lens mirror 1411 is reflecting the beam toward the angle 1401, but because of the misalignment, a portion of the laser is striking the edge of the ring/cap at area 1435 instead of passing cleanly through the aperture 1433. A sensor, or simply the observer noticing the interaction, can reveal this clipping. FIG. 12 thus highlights that the alignment hole 1433 in the ring allows the user to see when the laser beam 1450 is not perfectly centered: any contact of the beam with the ring (at point 1435) will scatter light, alerting the surgeon to adjust alignment. The figure shows that when properly aligned, the beam 1450 should pass through the aperture 1433 without hitting the ring, whereas here the misalignment causes a visible issue at 1435. This built-in feedback mechanism is a key safety and efficacy feature when using large-diameter laser beams.

FIG. 13 is a front view assembly diagram of the illumination ring 1430 and gonio lens 1410, illustrating their alignment. In this view, the eye 1400, lens 1410, and ring 1430 are shown in line with each other (as if looking head-on at the patient's eye). FIG. 13 demonstrates how the ring 1430 fits over the lens 1410, aligning its central opening with the gonio lens's optical axis and mirror. This figure emphasizes the concentric design of the ring and lens, which is important for ensuring the illumination uniformly surrounds the viewing area.

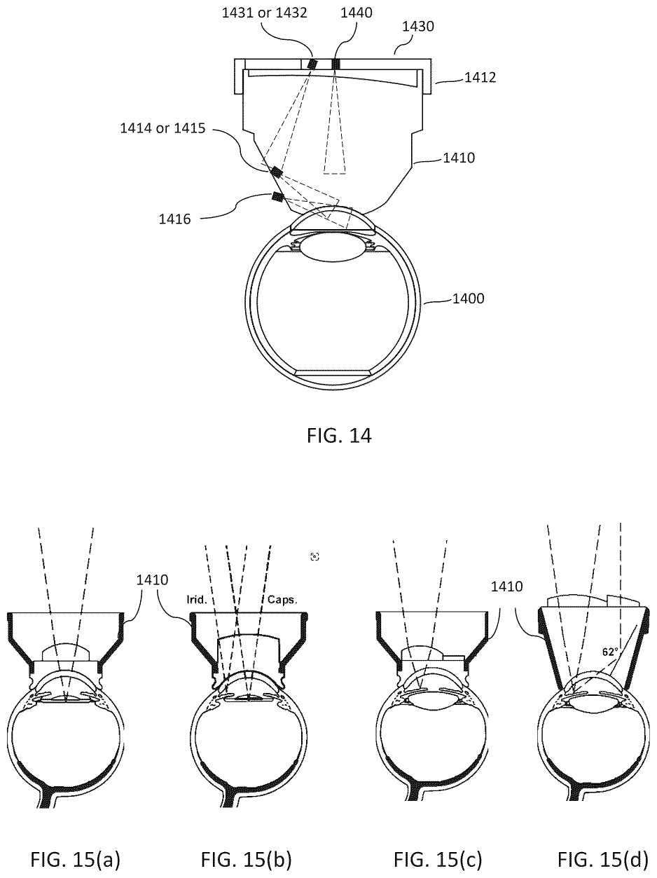

FIG. 14 is a cross-sectional side view of a gonioscopic lens assembly that combines multiple illumination modalities. The gonio lens 1410 on eye 1400 is shown with a top-mounted illumination cap 1430 (with its own lights 1431/1432 and a central element 1440) as well as additional built-in light sources 1414, 1415 located on or near the lens's sidewall 1416. In this embodiment, one or more LEDs 1414, 1415 are embedded in the lower portion or side of the lens and oriented to project light horizontally or obliquely into the anterior chamber. These additional lights 1414/1415 complement the overhead ring lights 1430, creating a multi-directional illumination system. Dashed lines in FIG. 14 indicate how light from the side LED 1416 can shine into the angle from a different angle (perhaps beneath the iris) while the top lights 1431/1432 shine from above. This comprehensive lighting reduces shadows and ensures that the target tissue is illuminated from various angles simultaneously. FIG. 14 corresponds to an embodiment described in the text where lateral or bottom-mounted illumination is used in conjunction with an overhead ring.

FIG. 15 shows various commercially available (Ocular Instruments) eye lenses that are typically placed and held by hand on the cornea with a flange on the outer rim for improved stability. Version (a) is a capsulotomy lens, (b) is a combination iridotomy/capsulotomy lens, (c) is an iridotomy lens, and (d) is a combination iridotomy and mirrored gonio lens.

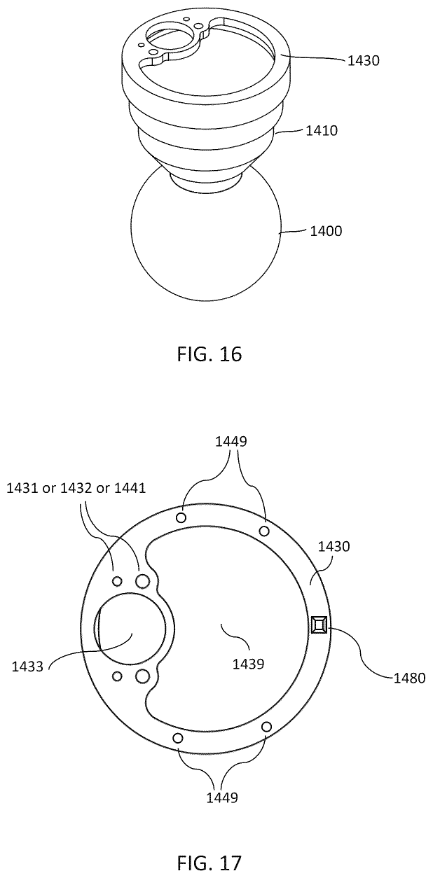

FIG. 16 is a perspective view of a gonio lens 1410 on an eye 1400 with a partially open illumination cap 1430. In this embodiment, the cap 1430 covers the left side of the lens but is open over the central and right side (visible as a large cut-out area 1439). An alignment aperture 1433 is present on the left side of the cap (a small hole through the cap), and a couple of integrated units 1431 or 1432 (which could be lights or sensors) are arranged around that aperture. FIG. 16 demonstrates how the cap 1430 is shaped more like a half-ring, providing illumination and alignment features on one side while leaving the other side open for direct access/viewing. This design is particularly useful for combined gonioscopic surgical procedures where instruments or implants need to be seen through the top of the lens (the open region 1439).

FIG. 17 is a top view of the illumination cap of FIG. 16. It clearly shows the ring 1430 structure with a significant open region 1439 on one side (right side in the figure) and a solid portion on the other side that carries the alignment hole 1433 and two lighting/imaging elements 1431 or 1432. The open region 1439 corresponds to the area above the lens that is not covered by the cap, allowing direct line-of-sight into the eye. FIG. 17 highlights the difference between a “light cap” (which covers more of the lens surface) and a “light ring” (which may only be a narrow band around the periphery). This figure matches the description of a configuration where the central and right regions are open, facilitating direct visualization of the iris and pupil.

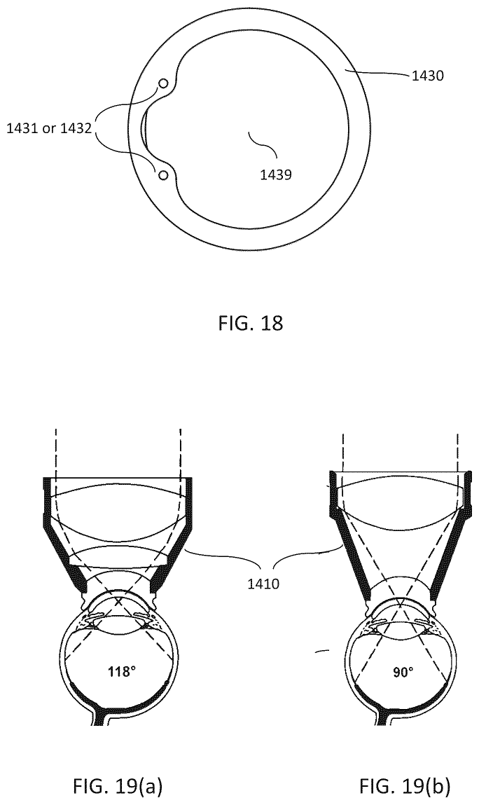

FIG. 18 is a top view of another illumination ring embodiment, showing a generally circular ring 1430 that runs around the outer edge of the lens, leaving most of the lens's top open (1439). In FIG. 18, the ring appears more symmetric, covering the perimeter while exposing the center. A pair of illumination elements 1431 or 1432 are indicated on one side of the ring (left side in the figure), but similar elements could be spaced around.

FIG. 19 shows two commercially available (Ocular Instruments) retina lenses with different wide-angle viewing and treatment access.

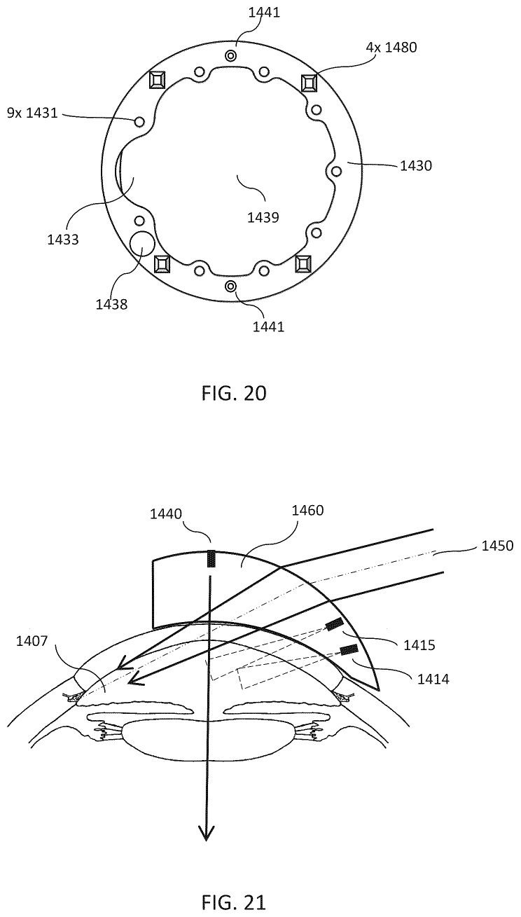

FIG. 20 is a top view of an illumination ring 1430 adapted for vitreoretinal or posterior segment applications. In this figure, the ring 1430 carries a multitude of illumination units (labeled as “9×1431” to indicate, for example, nine LEDs 1431 spaced evenly around the circumference). The central opening 1439 is large, as this ring might be placed on a wide-field contact lens used for retinal visualization. The multiple LEDs ensure panoramic illumination inside the eye, which can be particularly useful for stereoscopic viewing or highlighting translucent structures like vitreous strands or floaters. FIG. 20 corresponds to an embodiment for illuminating different depths in the vitreous body, where several lights can be modulated (in intensity or focus) to create a three-dimensional lighting effect that enhances contrast of floating opacities.

FIG. 21 is a cross-sectional side view of a direct contact lens 1460 (non-mirrored) with integrated illumination for both anterior and posterior segment viewing. In this illustration, the contact lens 1460 (which could be a dome-shaped lens for viewing the angle directly or the vitreous) is on the eye. The lens 1460 has embedded illumination sources 1414, 1415 at its periphery aimed into the anterior chamber angle (similar to earlier embodiments but without using a mirror). A central module 1440 is positioned on or above the lens, aligned with the pupil. The module 1440 may contain a camera or an additional light source. Dashed lines 1450 represent diagnostic or laser beams that are directed through the pupil into the eye (for example, targeting the trabecular meshwork or vitreous floaters). The figure shows arrows from the peripheral LEDs 1414, 1415 illuminating the angle region and possibly arrows from the central module 1440 indicating either illumination or viewing toward the retina. The iris in a normal and possibly constricted state (1407) is depicted, as in FIG. 11, to illustrate how this direct lens can also benefit from pupil management. FIG. 21 demonstrates an embodiment where the lens itself (1460) does not rely on an internal mirror but still achieves targeted lighting: some lights (1414, 1415) aim into the angle from the sides, while the central axis can be used for through-pupil observation or treatment.

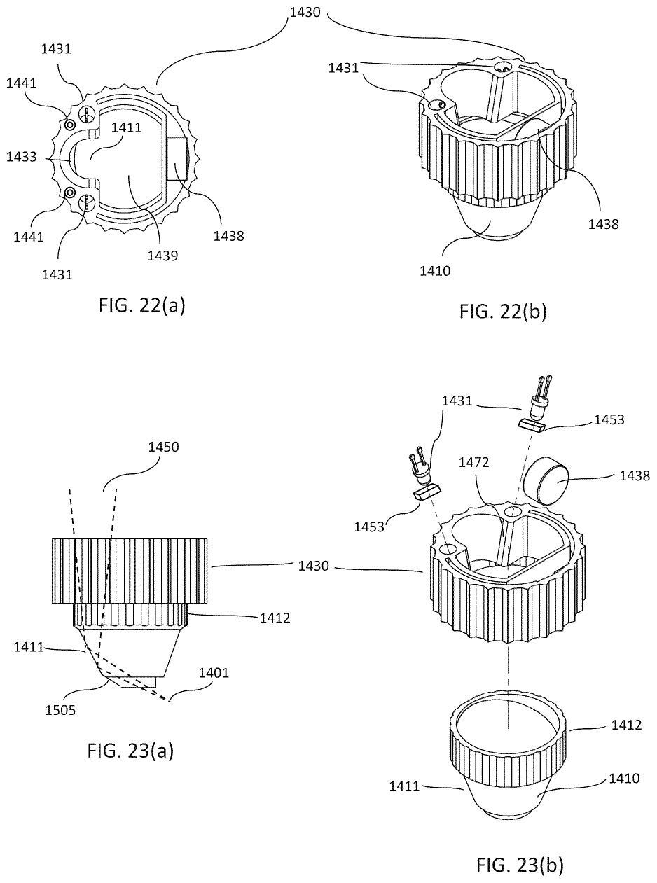

FIG. 22(a) is a top perspective view of a single-mirror gonioscopic lens 1410 outfitted with an illumination ring 1430. The ring shows multiple LED modules 1431 arranged around a central opening 1433 that is aligned with the lens's internal mirror 1411 (the mirror is visible through the opening). Also aligned around the mirror are two cameras 1441 that can create a 3D visualization of the anterior angle of the eye via the mirror. This configuration forms a halo of light and imaging around the mirror without obstructing the viewing path through the lens. A small battery 1438 is integrated into the ring to power the LEDs and cameras, making the unit entirely self-contained. The cameras 1441 transmit the image data to a system computer via a cable or a small wireless component integrated in the ring. FIG. 22(b) provides a top-front perspective of the same assembly, illustrating how the illumination ring 1430 securely mounts over the lens's upper rim 1412. The ring's compact design and central aperture ensure that the lens's optical pathway remains clear while the surrounding LEDs 1431 can brightly illuminate the eye's anterior chamber angle via reflection off the mirror 1411.

FIG. 23(a) is a side elevational view (partially in section) of the gonio lens 1410 with the illumination ring 1430, highlighting the tilted orientation of the LED channels 1472. The LED channels 1472 within the ring are angled inward such that light emitted from the LEDs 1431 is directed toward the central mirror 1411 and reflected into the anterior chamber angle region 1401. A dashed line in this view indicates the angled light path from a ring LED to the mirror. FIG. 23(b) is an exploded perspective view of the same illumination ring assembly. In FIG. 23(b), the ring 1430 is shown separated from the gonio lens 1410, and its internal components are illustrated: the coin-cell battery 1438 is removed from its compartment in the ring, and the LED modules 1431 (which normally sit in the tilted channels 1472 of the ring) are depicted above their mounting positions. This exploded view demonstrates how the ring attaches to the lens 1410—the ring is designed to snugly fit over the lens's top flange 1412, aligning its LEDs with the mirror 1411 so that, when assembled, the light channels 1472 precisely target the mirror.

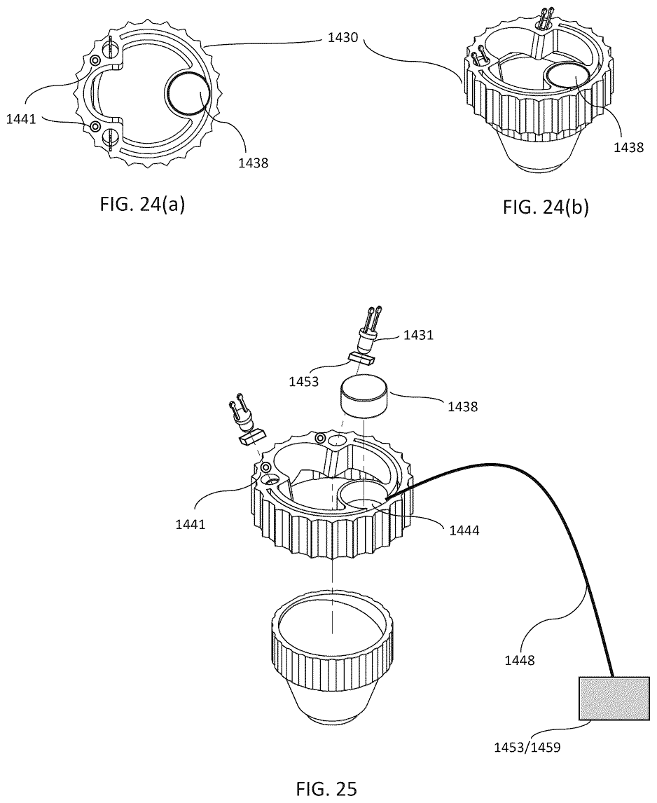

FIG. 24(a) and FIG. 24(b) illustrate a more compact variant of the illumination ring for a gonioscopic lens, featuring a horizontally oriented battery. FIG. 24(a) is a top view of this variant ring 1430, showing a coin-cell battery 1438 lying flat within the ring's housing. By positioning the battery in a horizontal orientation, the ring's overall height is reduced, resulting in a lower-profile attachment on the lens. FIG. 24(b) is a perspective view of the assembled compact ring mounted on the gonio lens 1410. This design maintains a central aperture for the mirror and uses a similar arrangement of LEDs as in FIG. 22, but the battery placement is reconfigured to be flat, making the illumination module more streamlined while still directing light effectively to the mirror 1411.

FIG. 25 provides an exploded view of the compact ring assembly of FIG. 24. In this illustration, the flat battery 1438 is shown removed from the ring housing 1430, and the gonio lens 1410 is depicted below. The LED elements (not separately numbered in this figure) are housed in the ring in the same manner as the earlier embodiment, positioned to align with the lens's mirror inside. The exploded view highlights the simplified, low-profile construction of this variant: the battery 1438 nests into a recess in the ring 1430 from above, and the ring attaches onto the lens 1410 to provide illumination in the same fashion as described for FIGS. 22-23 (directing light to the mirror and into the anterior angle). The illumination module FIG. 25 also includes 1 or multiple mini-cameras 1441 embedded in the ring and oriented to provide imaging into one or multiple areas of the eye.

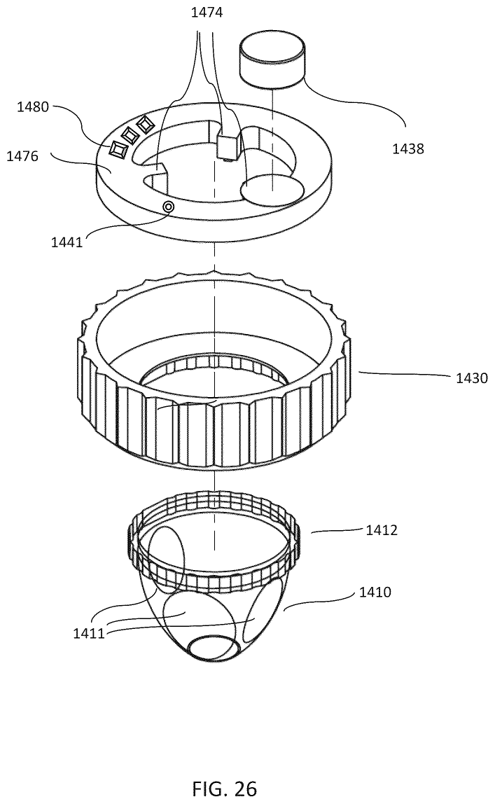

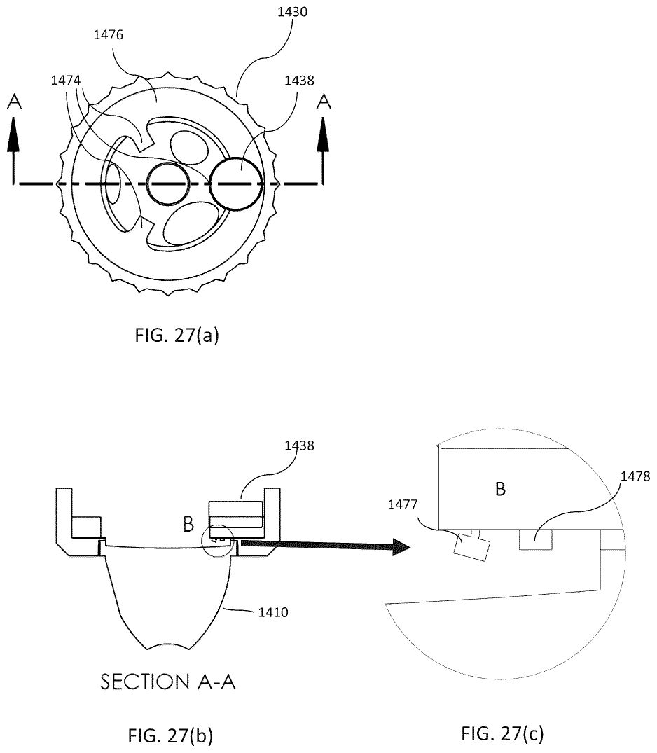

FIG. 26 is an exploded view of an illumination ring assembly adapted for a three-mirror diagnostic contact lens. In this figure, the ring's components are shown separated above the three-mirror lens 1410. The main ring body 1430 (with a grooved outer surface for grip) sits below a top insert piece 1476 that carries the LED light sources. The insert piece 1476 can double as an electronics PCB board that contains all control electronics. On the underside of this insert piece 1476, a set of inward protrusions 1474 are placed between the 3 mirrors. Each protrusion 1474 houses two LED modules (see FIG. 27(a)-27(c)), 1477 and 1478, positioned to shine light onto the adjacent mirror. Thus, when the ring is assembled onto the lens, each of the lens's three mirrors will have a dedicated light source aimed at it from each side above. The ring's power source, a coin-cell battery 1438, is also shown removed from its slot in the ring. At the bottom of the exploded stack, the three-mirror lens 1410 itself is depicted with its upper flange 1412 and one of its internal mirror surfaces 1411 visible. This exploded view illustrates how the illumination ring aligns over the lens 1410 such that the protrusions 1474 position the LEDs 1477 and 1478 to directly illuminate each corresponding mirror inside the lens. The illumination module FIG. 26 also includes 1 or multiple optional mini-cameras 1441 embedded in the ring and oriented to provide imaging in to one or multiple areas of the eye. Such a camera(s) can be optionally included in all illumination module embodiments throughout this disclosure. All camera modules throughout the disclosure can be powered by the on-board battery(ies) and transmit their imaging data to a receiver module via wireless communication or can be powered by wire and/or transmit their imaging data via wired connection to a computer/electronics module (see FIG. 31).

FIG. 27(a)-27(c) shows the illumination ring in place on a three-mirror lens and includes a cross-sectional detail of the light coupling to a mirror. FIG. 27(a) is a top view of the illumination ring 1430 mounted on the three-mirror contact lens 1410. The ring's central opening reveals the three internal mirrors of the lens arranged at intervals around the interior. Each inward protrusion 1474 lays in between two mirrors. The ring's battery 1438 is visible on one side and functions as one of the 3 protrusions as well. FIG. 27(b) is a cross-sectional side view taken along line A-A of FIG. 27(a), cutting through one of the lens's mirrors on the left and its aligned illumination module. In the section view of FIG. 27(b), an enlarged detail callout “B” is provided in FIG. 27(c) to highlight how the light is directed. In the magnified detail B in FIG. 27(c), each of the light sources 1477 and 1478 is aimed at different adjacent mirrors. This arrangement is duplicated for each of the three mirrors in the lens, so that each diagnostic mirror receives its own targeted illumination from both sides along that mirror's viewing axis, greatly improving the visibility through all angles of the three-mirror lens.

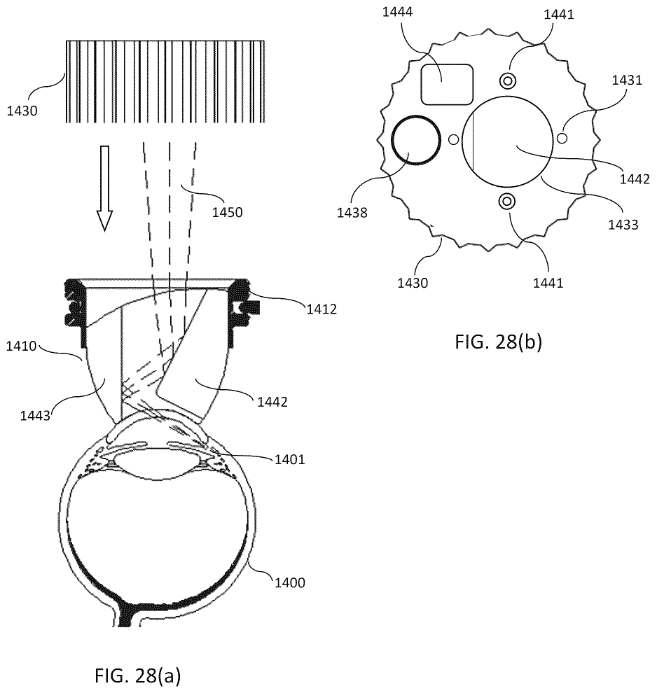

FIG. 28(a) shows an embodiment with the illumination ring 1430 floating over a double mirror gonio lens 1410. As an example, shown here is the Ocular Ahmed DVX Surgical Gonio lens. It has a two-mirror design 1442 and 1443 that avoids the flipped image of a single mirror gonio lens design. The illumination ring 1430 is mounted over the outer rim 1412 of the gonio lens 1410. The entry for the imaging or laser treatment beam 1450 is vertical and somewhat shifted from the overall center. This gonio lens is used to provide visual or treatment access to the anterior angle 1401 of the eye 1400. FIG. 28(b) shows a top view of the illumination ring 1430 with the access hole 1433, the battery 1438, two cameras 1441, two light sources 1431, and an electronics and optional wireless module 1444.

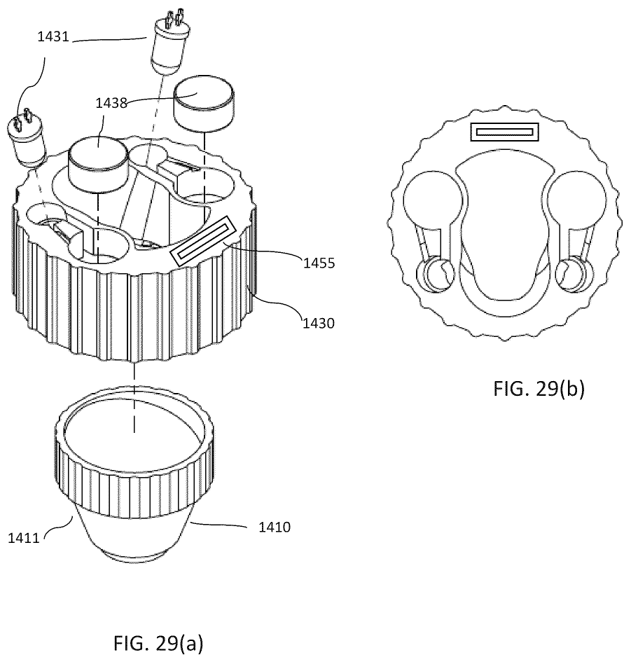

FIG. 29(a) shows an exploded view of another embodiment of an illumination-gonio lens module with two LEDs 1431, powered by two batteries 1438 with the LEDs emitting a narrow spot light beam that is angled such that a significant part of the light cone is directed towards the mirror section 1411 of the gonio lens 1410 and from there reflected to the target illumination zone int the anterior angle area of the eye (in this case being underneath the opposite side of the mirror 1411. This illumination module 1430 also includes a control electronics module 1455 which can consist of simple resistors to drive the LEDs up to a full micro controller unit that allows independent full brightness control for all LED units, illumination timing and cutoff (for low battery), pulsed/modulated illumination light control, wavelength selection/switching, status indication, embedded RFID implementation, pressure (against eye) sensing and visual and/or audio feedback signaling and wireless data communication to an external computer FIG. 29 (b) shows a top view of (a).

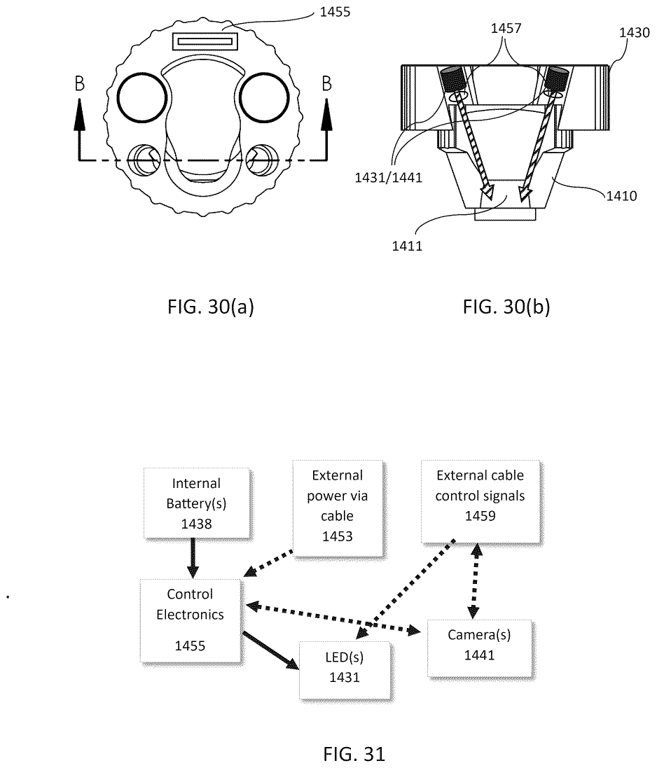

FIG. 30(a) shows a top view of another embodiment of an illumination module 1430 (similar to FIG. 29, but with a slimmer design) with an indicated sectional view B that is shown in FIG. 30 (b). Here the LED 1431 or Camera 1441 modules (or one each) can be seen mounted with an angle such that the view/light beam is pointing towards the gonio mirror 1411. Also shown here are optional focusing/imaging optics 1457.

FIG. 31 shows an illumination system block diagram. Several embodiment configurations are illustrated here. For example, the Illumination module could consist of only an internal battery 1438, a control electronics module 1455 and the LEDs) 1431, or the same but being powered by an external power source via a cable 1453 instead of the battery(s) 1438. The module can also include 1 or multiple cameras 1441 that are either controlled by the internal control electronics unit 1455 (and transmit the video signal via wireless to an external computer) they are connected to an external computer via an external control cable signal link 1459. External power can be provided via a cable to the internal control electronics unit 1455 which then controls and drives the LED(s) and/or camera(s), or external control cables 1459 (including power) can directly power and control the LED(s) and/or camera(s) and thereby eliminating the need for an internal control electronics unit 1455.

Polarization Capabilities: In some embodiments, the illumination modules (whether as a detachable ring or integrated into the lens) include a small linear polarizer element in front of each LED. The emitted light is thus polarized, and a complementary adjustable polarizer (an analyzer filter) may be provided in the observing microscope or camera. By adjusting the relative polarization angle (for example, cross-polarizing the illumination relative to the viewing optics), the system can dramatically reduce glare from ocular surfaces and enhance the contrast of tissue structures. This optional feature allows improved visualization of details in the trabecular meshwork, retina, or other target tissue by filtering out specular reflections, and it can be engaged as needed without altering the illumination device's placement on the eye.

Integrated Gonioscopic Lens Illumination (Single-Mirror Embodiment): FIGS. 1-3 illustrate a gonioscopic lens 1410 with integrated illumination sources. The gonio lens 1410 is a contact lens used for viewing the anterior chamber angle 1401 (where the trabecular meshwork and other angle structures reside) by means of an inclined mirror 1411. In a conventional use, this lens would be held against the patient's cornea 1405 and the surgeon would look through the top while an external slit lamp beam is directed into the mirror. In some embodiments can be configured to, the gonio lens 1410 itself carries miniature LED lights 1414, 1415 near the mirror, thus internalizing the illumination. As shown in FIGS. 2-3, the LEDs 1414 are embedded around the mirror surface 1411 within the lens material. They are positioned such that they do not obstruct the mirror's reflective function (leaving an unobstructed central portion of the mirror). Each LED 1414, 1415 is angled (both inward toward the optical axis and downward toward the eye) to shine light onto the target tissue that the mirror 1411 is viewing.

When the gonio lens 1410 is placed on the eye and one of the angle structures (e.g., trabecular meshwork) is being observed through mirror 1411, the corresponding LEDs automatically illuminate that structure. Because the illumination is originating right at the lens, it follows the lens's orientation. For example, if the surgeon rotates the lens slightly to bring another segment of the angle into view, the integrated lights rotate along with it and continue to light the new segment. This is a stark contrast to an external slit lamp illumination, which would need to be repositioned for the new angle. The integrated approach therefore eliminates the need for continuous re-adjustment of the light source during procedures like SLT or gonioscopic laser treatments. The surgeon can maintain one hand on the lens and the other on the laser or other instrument, confident that the illumination is locked onto the viewing area. Using an illumination ring on a two-mirror gonio lens, such as that shown in FIG. 28, provides the same features as a single-mirror gonio lens, except for the image being upside right due to the two-mirror reflection and the shifted entry point for the laser/visualization beam.

Power for the LEDs in the gonio lens can be supplied in various ways. In some embodiments, a thin flexible cable runs from the lens 1410/1430 to a small external battery pack or connector; the cable could be attached along the instrument holding the lens to minimize interference. In preferred embodiments, however, the power source is miniaturized and made part of the lens assembly itself, such as a tiny coin cell or rechargeable battery 1438 embedded in the lens or in a detachable cap. FIG. 9, for instance, shows a battery 1438 integrated into the illumination cap 1430. For an all-integrated gonio lens (without a separate cap), a similar approach can be taken: the lens body 1410 can have a built-in battery in a widened portion of its periphery or handle 1412. This self-contained power enables the lens to be completely cordless. One or multiple microswitch(es) or button(s) on the lens or illumination ring cap (e.g., 1480 in FIG. 9, FIG. 17, or FIG. 26) can allow the surgeon to turn the illumination on/off, adjust brightness, switch wavelength, illumination mode or mini-camera settings before or during the procedure.

The light emitted by the LEDs 1414, 1415 can be adjusted in intensity to avoid overwhelming the eye. Because the LEDs are so close to the target, even a small, low-power LED can provide sufficient illumination. This reduces the risk of patient discomfort since high intensity light is not flooding the entire eye, only the region of interest. Additionally, since the light is incident at an angle matching the mirror view, less stray light enters the pupil—contrast FIG. 4, where external light 1420 sent through the cornea partly goes into the pupil (1422) causing glare on the retina 1406. In some embodiments, the majority of light from LEDs 1414 hits the angle and is reflected back out, or absorbed by the target tissue, rather than going through the pupil.

Multiple Illumination Sources and Wavelengths: Using more than one LED (as shown in FIG. 2 with four LEDs) allows for a uniform and shadow-free illumination. When two or more LEDs 1414, 1415 are arranged symmetrically around the mirror, their light overlaps at the target, minimizing any shadows that one light alone might cast. Moreover, having multiple sources permits mixing different wavelengths. One embodiment uses pairs of LEDs of different colors—for example, one white light and one green light. The surgeon could activate the white LEDs for a general view, then switch to green or red illumination mode to enhance contrast of certain structures. Infrared (IR) LEDs could also be included for use with IR cameras to visualize structures behind the iris or within the scleral wall (like collector channels) that are not seen under visible light. Because all these LEDs are built into the lens or illumination ring, switching between them is instantaneous and does not require replacing any equipment or filters; it can be done via an electronic control.

Reusable vs. Disposable Lens Options: The gonioscopic lens assembly 1410 with integrated LEDs can be made either reusable (constructed from durable, sterilizable materials like medical-grade glass or polycarbonate) or as a low-cost disposable. In accordance with some embodiments, miniature LEDs and small batteries can enable the creation of a single-use gonio lens with built-in illumination, in a manner that is economically feasible. In a disposable version, the entire lens (or at least the lighting components) would be discarded after one use, ensuring perfect cleanliness and performance each time. In a reusable version, the lens must be sterilized, but the LED components can be sealed to withstand disinfectants. Some embodiments can be configured in either or both variations. Notably, even the disposable implementation provides high-quality optics—materials like acrylic or PMMA could be used to mold the lens for one-time use.

Integration of Imaging Sensors: In some embodiments (as suggested in FIG. 9 and its accompanying description), one or more miniature cameras or optical sensors are integrated into the gonio lens assembly alongside the LEDs. These can capture the surgeon's view or even see wavelengths beyond human vision (like IR). For example, a tiny camera 1432 could be placed just above the mirror 1411 inside a transparent portion of the lens or in the illumination ring, looking into the angle. It would have the similar perspective as the surgeon's eyepiece (or the laser's path). This camera could feed live video to a screen, allowing for heads-up display or recording. When using an IR LED to visualize structures, an IR-sensitive camera would pick up details the surgeon might not see directly. Multiple sensors can also enable 3D depth perception or wide-angle coverage. The data from these sensors might be used by an auto-guidance system to assist in laser aiming: for instance, image processing could detect the trabecular meshwork and adjust the laser position accordingly. Such advanced features are optional, but demonstrate the platform nature of some embodiments of the devices disclosed herein—the contact lens assembly becomes not just a passive optical element but an active smart device.

Applicability to Multi-Mirror Lenses: While much of the above description focuses on a single-mirror gonioscopic lens, some embodiments can be configured to comprise contact lenses with multiple mirrors, such as the classic three-mirror diagnostic/therapeutic lens. A three-mirror lens typically has three angled mirrors inside a single contact lens body, each angled differently to visualize different parts of the eye (one for the iridocorneal angle, and two others for retinal views at various orientations). In some embodiments, a three-mirror lens device is equipped with integrated illumination as well. For example, small LEDs can be placed adjacent to each of the three mirrors, tuned to illuminate the field of view of that particular mirror. The mirror meant for gonioscopic (angle) viewing could have an LED similar to the one described for the single-mirror gonio lens. The mirrors for retinal views (which are angled differently, e.g., 67° and 59° mirrors) could have their own small LEDs directing light to the peripheral fundus when those mirrors are in use. Alternatively, an illumination ring/cap, as shown in FIG. 26 and FIGS. 27, 1430 could be adapted to fit over a three-mirror lens 1410, with multiple lights 1477 and 1478 arranged to correspond to the multiple viewing sectors. By implementing this, a three-mirror lens used for diagnosis or laser photocoagulation can have built-in 360° illumination, improving visualization of the fundus without needing an indirect ophthalmoscope or external light source. When the user rotates the three-mirror lens, the integrated lights rotate accordingly, always staying in sync with the mirror that is being looked through at that moment. This can be extremely useful, for example, in pan-retinal photocoagulation lasers or peripheral retina examinations, where consistent lighting of the retina through the mirror is needed for each spot without repeatedly adjusting an overhead lamp.

Illumination Ring/Cap Attachment (General): FIGS. 6-9, 13-20, and 22-27 illustrate embodiments where the illumination system is part of a ring or cap 1430 that attaches to a contact lens (such as gonio lens 1410). This ring can be thought of as an accessory or retrofit component that can be coupled to or used with existing devices to provide benefits to those existing designs. In some embodiments, the ring 1430 is a thin circular structure that snaps onto the top of a gonio or other contact lens. It carries a set of lights 1431, 1432, 1477, 1478 distributed around its circumference, as well as possibly other components like a central light/camera 1440, a battery 1438, status indicators 1437, and microswitches 1480 (as detailed, e.g., in FIG. 9).

Once mounted, the ring's lights surround the lens's viewing aperture. They are positioned outside the central optical zone for the particular treatment, so they do not obstruct the surgeon's view or the passage of a laser beam through the lens. The aperture 1433 of the ring aligns with the mirror or optical path of the underlying lens, effectively forming a halo of illumination around that path. This design ensures that even large-diameter beams can pass without clipping, as there is a clear hole directly above the mirror (see FIG. 12 and corresponding description). The ring thus provides an aligned light field that travels with the lens.

Advantages in Laser Alignment: The ring embodiment is particularly advantageous for high-precision laser procedures. As shown in FIG. 12, the ring 1430 can incorporate an alignment aperture 1433 and function as a guide to ensure the laser beam is centered. For instance, during a trabecular meshwork laser treatment with a femtosecond or picosecond laser, the surgeon can observe the reflection of the aiming beam through the aperture. If the beam starts to clip the edge (area 1435), the illuminated or shadow pattern on the aperture immediately signals misalignment, prompting correction before firing the high-power laser. This feature addresses the earlier-discussed problem of large-NA beams: with the ring's guide, the surgeon is effectively given a built-in targeting reticle. Some embodiments may include a sensor or photodiode at the aperture edge that detects laser light contact and could even produce an electronic alert (or trigger indicator LEDs 1437 to flash) if misalignment is detected. In any case, maintaining the full, unobstructed laser beam path is ensured by the mechanical design of the ring.

Status Indicators and Multi-Functional Feedback: The illumination ring 1430 may include small indicator LEDs 1437 (FIG. 9) that communicate statuses to the user. For example, an indicator might glow green when the device is powered on and within safe intensity limits, or blink red if the battery is low. Another indicator could be linked to a microswitch or sensor that detects the laser's ready status from the laser system (by wire or wirelessly) and then show a blue light when the laser is armed. These indicators help integrate the lens-based illumination system into the surgical workflow by providing at-a-glance information right at the lens. Additionally, markings or etched patterns on the ring can serve as orientation guides. For example, a mark could indicate the mirror position under the cap, aiding the surgeon in orientation of the lens. All these additions further reduce the surgeon's cognitive load, keeping relevant info within the field of view.

Central Illumination/Imaging Module: Some embodiments feature a central element 1440 mounted on the ring or cap (as seen, e.g., in FIGS. 9-11). This element 1440 can be a downward-pointing light (such as an LED or a laser diode) or a tiny camera. When it is a light, it is aimed straight through the patient's pupil toward the retina 1406 (FIG. 11). One use of this central light is to intentionally induce the pupillary reflex to constrict the pupil, as explained earlier. By momentarily shining a controlled light onto the retina, the pupil contracts (iris 1407), which has the benefit of opening up the angle for treatment. In other cases, the central light 1440 could serve as a coaxial illuminator for directly viewing the posterior segment (like a mini flashlight into the eye's interior), which could be helpful if the practitioner briefly wants to switch from angle work to checking the fundus without changing instruments.

If the central element 1440 is a camera or sensor (with an optional LED as well), it can capture a direct axial view. In a gonioscopic context, that might be used to align the gonio lens centrally over the pupil or to monitor pupil size. In a vitreoretinal context, such a camera might record images of the retina while the ring's peripheral lights 1431/1432 or a central light next to the camera illuminate it. The presence of a central module is optional and may not be used in every procedure, but the design accommodates it.

Open Ring Designs for Special Applications: FIGS. 16-19 show that the illumination device can take the form of a partially open ring or segmented cap. This is useful in surgeries where, in addition to using the mirror, the surgeon needs to look directly into the eye or insert instruments. For example, in MIGS (minimally invasive glaucoma surgery) or angle procedures where a tiny stent or device is implanted into Schlemm's canal, the surgeon often uses a gonio lens but also watches part of the procedure directly through the cornea (outside the mirror view) to ensure the implant is properly positioned. A light ring 1430 that only covers a portion of the lens (leaving region 1439 open) provides the necessary illumination on the mirror side (viewable via aperture 1433), while allowing an unobstructed view through the uncovered portion. In FIG. 17, for instance, the right side of the lens is open (1439) so the surgeon can directly see instruments in the anterior chamber, whereas the left side has the lights focusing on the angle via the mirror under the opening 1433. This configuration maximizes versatility—practically giving the effect of both an illuminated gonio lens and a clear surgical lens in one. Additional optional lights 1449 in the outer perimeter allow for general or central illumination.

Office-Based Mixed Procedures and Surgical Access: In accordance with some embodiments, the partially open or segmented illumination configuration (as illustrated in FIGS. 12 and 23(a)-(b) is specifically adapted to facilitate office-based “mixed procedures,” such as the intracameral delivery of sustained-release drug implants (e.g., iDose®, Durysta®) or Minimally Invasive Glaucoma Surgery (MIGS). Conventionally, these procedures face the “three-hand problem” (when performed at a slit lamp or Opthalmoscope, Lupe or other office setting): a surgeon needs one hand for the gonio/contact lens, one for the slit lamp or other illumination setting, and a third for the injector. The disclosed self-illuminated device resolves this by eliminating the slit lamp/other illumination part. The patient may be placed in a supine or semi-reclined position (e.g., 45 degrees). The surgeon holds the illuminated lens 1430 with one hand, maintaining a view of the angle 1401 via the integrated lights 1431. Using a gonio lens with a cornea access notch 1505 behind the mirror part for example FIG. 1, or FIGS. 12, 23(a)), 1505, allows surgical access to the eye. Such an open structural gap allows partial access to the edge of the cornea 1505, for example, to provide physical clearance for a drug injector or surgical instrument to traverse the anterior chamber from the temporal side and access the nasal angle (or vice versa) without colliding with the gonio and illumination hardware.

Direct Gonio Lens with Multi-Directional Illumination: In FIG. 21 (and corresponding text), a direct gonio lens 1460 embodiment is shown. Unlike a mirrored gonio lens, a direct gonio (such as a Koeppe lens) gives a view of the angle by looking directly (often used in supine patients or with an ocular imaging system). In accordance with some embodiments, the direct lens 1460 is augmented with lights 1414, 1415 that project into the angle from around the lens periphery, and possibly with a central module 1440 on top. The result is that the angle can be illuminated without a mirror, allowing direct observation, imaging or treatment. Simultaneously, the central module could be used for viewing through the pupil (for example, to monitor the effect of the treatment or to get a direct view of the retina without changing lenses). Such an arrangement could be useful in certain laser procedures where both angle and axial views are needed. One example might be combined procedures where a laser is applied to the angle (e.g., laser goniopuncture after canaloplasty) and the central pupil is observed for eye tracking. The direct lens with integrated illumination acts as a multipurpose platform, providing comprehensive lighting whether one is looking at the front or back of the eye.

The illuminated gonio lens 1410 with the illumination ring 1430 (e.g., as shown in FIG. 23(b)), can be made either reusable or disposable. Reusable versions may be constructed from rigid materials like glass, sapphire, or durable plastics (e.g., PEEK or polycarbonate) that can withstand autoclave or chemical sterilization. Disposable versions can be made from molded PMMA (polymethylmethacrylate) or polycarbonate that are cheaper to produce. The cost of the included LEDs 1431 and electronics is kept low by using mass-produced mini-LED components and simple circuits, so even a single-use lens is economically feasible. As noted in the provisional disclosure, integrating a few LEDs adds minimal cost, enabling both reusable and one-time-use configurations. Preferred sterilization methods for the reusable devices include ethylene oxide gas (EtO) and hydrogen peroxide plasma, which effectively sterilize without damaging sensitive electronics (these methods are commonly used for medical devices that cannot tolerate high-heat autoclaving). The materials selected (for example, medical-grade polycarbonate like PC-ISO or a biocompatible polymer like PEEK) are chosen for both optical clarity and sterilization compatibility.

Optionally, miniature cameras or sensors can be integrated into the gonio lens assembly along with the LEDs. As described in several embodiments (e.g., FIG. 14, FIG. 20, FIG. 22(a), and FIG. 28), cameras as small as 0.3 to 8 mm diameter (preferably 1 to 3 mm) can be placed at or near the mirror to capture the view directly. In practice, one might embed such a tiny mini camera adjacent to the mirror 1411 (or 1442 in FIG. 28), looking near the same optical window. This camera could transmit video to a monitor, allowing for digital viewing or recording of the angle, or even automated image analysis. Furthermore, using two spatially separated cameras on both sides of the mirror (e.g., 1441 in FIG. 28 or FIG. 22(a)-22(b)) and with a viewing angle tilted towards the mirror, will enable 3D imaging recording or live 3D diagnostic to determine the momentary z-focus alignment of the laser focus and thereby creating a tracking and adjustment signal to guide the operator for laser alignment or to automatically adjust the laser focus position. The synergy of on-board illumination and imaging means the device could function as a standalone diagnostic tool—for instance, capturing images of the angle before and after a laser treatment, or enabling telemedicine where a clinician can remotely observe the angle via the device's camera feed.

Heads-Up Display and Ergonomics: To further enable office-based interventions, the integrated camera modules 1441 (shown in FIGS. 9, 11, 20, 22, 25 or FIG. 31) facilitate a “heads-up” surgical workflow. Rather than bending over a slit lamp or viewing through a lens eyepiece, the physician views a high-definition external monitor or uses a virtual reality headset or augmented reality (AR) glasses connected to the device (wirelessly or via cable 1459). The camera system may feature 3D and auto-focus capabilities centered on the illuminated target zone. This allows the physician to sit comfortably and guide the gonio lens with one hand and the therapeutic injector with the other, utilizing the on-screen image for precision placement. This setup effectively converts a standard examination room into a minor procedure suite.

Another optional enhancement for some embodiments of the gonio lens is the inclusion of an alignment aperture and safety features. Especially during laser procedures (like photodisruptive laser treatments of the Trabecular Meshwork or laser iridotomy), it is critical that the therapeutic laser beam not be obstructed or misaligned by the device. Some embodiments feature a central hole or transparent window through the lens or illumination cap that is aligned with the optical axis of the targeting path. This aperture (1433 in some figures) allows an aiming beam or the therapy laser to pass through uninhibited. If the gonio lens is slightly decentered, part of the laser's aiming beam might impinge on the opaque portion of the cap; by designing an aperture and perhaps a contrasting ring around it, the user can visually detect beam clipping. For example, a red alignment laser beam (which optionally could be made larger than the treatment beam at the aperture 1433) would appear cut-off if the device is misaligned, meaning the aiming beam will be partially visible (the clipping part) on part of the edge of the aperture 1433. The user can then reposition until the beam is fully clear through the aperture, ensuring safe delivery of the therapeutic laser. FIG. 9 or FIG. 28(b) illustrates an embodiment of an illumination cap 1430 on a gonio lens, which includes an alignment guide hole 1433. As shown in FIG. 6, the illumination cap/ring 1430 fits over the outer rim 1412 of the gonio lens 1410 and as shown in FIG. 9 contains LED units 1431 (and optionally sensor or camera units 1432). As shown in FIG. 12, a central opening 1433 is provided above the gonio mirror 1411. A laser beam 1450 can be directed through this opening; if the beam begins to hit the edge (at point 1435) it indicates misalignment. This design helps avoid situations where the therapeutic laser beam might accidentally hit the device or deliver an off-target shot. The cap can be detachable, allowing the gonio lens to be used with or without illumination as needed. The user interface lens/gonio lens 1410 can be reusable while the illumination cap/ring 1430 is disposable or vice versa, or both being reusable, or both being disposable. In alternative designs, the cap 1430 could be permanently integrated into the lens body, essentially forming an illuminated gonio lens as a single unit, reusable or disposable.

Illumination Ring Embodiments with Horizontal/Vertical Battery Orientation: Some embodiments can provide two notable embodiments of illumination rings for gonioscopic lenses, distinguished by battery orientation. These rings are external attachments that encircle or clip onto an existing gonio lens (or similar contact lens/patient interface). They serve to retrofit standard lenses with illumination and imaging capability.

Structured Light Angle Biometry: To objectively quantify the “openness” of the anterior chamber angle (e.g., Shaffer grading), the illumination system may utilize a structured light modality. Referring conceptually to FIG. 11, the light source (e.g., module 1440 or specific LEDs 1431) comprises a red laser diode or slit projector configured to emit a beam 1436 of known geometry (e.g., a light sheet with a vertical height of approximately 400 μm) at a fixed angle. The integrated camera 1441 captures the incidence of this beam on the angle structures. If the angle is open, the full beam height is visible on the trabecular meshwork. If the angle is narrow, the iris root “clips” or obstructs the lower portion of the beam. The system calculates the ratio of the visible beam to the obstructed portion to generate a quantitative “Angle Openness Score,” replacing subjective estimation with optical metrology.

In some embodiments, the battery is a thin cell (for example, a small coin cell battery mounted in a horizontal orientation relative to the ring (see for example FIG. 24 and FIG. 25) or vertically integrated (see for example FIG. 22(a)-22(b) and FIG. 23). One or multiple batteries can be used to power the one or multiple LEDs, camera(s) and additional control electronics that is included in the illumination module. The battery(ies) has (have) a preferred size of 1 mm to 8 mm thickness and 4 mm to 23 mm diameter (more preferred 1 mm to 5 mm thickness and 6 mm to 12 mm diameter) and is/are preferably shaped as a cylinder round cell type battery, but any other shape is possible as well. The battery(ies) is(are) single use or rechargeable. In order to achieve sufficient illumination on the target tissue one or multiple LED modules with sufficient brightness output are chosen. They come in various shapes and packaging form factors with or without focusing optics and a typical operating current of 5 mA to 25 mA per LED. In some cases, it is desirable to overdrive the LED's up to 100 mA per LED which significantly reduces the lifetime of such an LED, but is mitigated and a nonissue when the illumination module is a single use disposable item where a 10 to 100 minute lifetime is sufficient to last through any expected treatment time. To power a LED with a current requirement of 10 mA to 100 mA with a small battery also reaches and exceeds small battery power/current specification limits. Most small cell type batteries are not able to provide any current beyond a few mA. Some small Li-Ion or similar batteries that can provide a current of 10 mA or more are here preferably used throughout this disclosure. Furthermore in some embodiments one or multiple such battery(ies) (for example a LIR 1040 battery) is intentionally overdriven to achieve a current output of greater than 20 mA (preferably 25 mA to 100 mA) to power (overdrive) one or multiple bright LED(s) and thereby drastically reducing the operating time of such a battery to 5 to 60 minutes and significantly reducing its lifetime such that it is preferably only intended for single use.

The illumination output and features of these rings are similar to the integrated lens embodiment: multiple brightness levels, possibly different color LEDs, and the option for polarizing elements. Because these rings can be add-ons, they can be sold or provided as accessories to existing diagnostic lenses. A surgeon could attach the ring to a favorite gonio lens for a procedure requiring enhanced lighting, and then detach it for cleaning or when not needed, or discard after single use.

In some designs, the ring not only carries LEDs but can also support modular attachments—for example, a slot where a small camera module can be snapped in, or a port for plugging in external power if the battery is drained. The ring could have a standardized diameter or adjustable clasp to fit various lens brands (since gonio lenses come in slightly different diameters).

Crucially, both ring embodiments are engineered to ensure no significant obstruction of the physician's view. The rings sit at or just above the top surface of the lens and have a central opening that is at least as wide as the lens's needed optical aperture.

Illumination for Three-Mirror Diagnostic Lenses: Three-mirror lenses (such as Goldmann-type diagnostic lenses, see FIG. 26 and FIG. 27) present unique illumination challenges, as each mirror views a different region of the interior eye at a different angle. Some embodiments can be configured to include an illumination ring tailored for these lenses. The three-mirror lens typically has a large contact surface and three angled mirrors spaced roughly 120° apart, plus a central view through the lens. For reference, a standard Goldmann lens has mirrors angled about 59°, 67°, and 73° from the eye's optical axis, allowing views of the far peripheral fundus, mid-peripheral retina, and anterior chamber angle, respectively.

The ring for the three-mirror lens can be designed to not obstruct the central view (the direct view used to see the posterior pole). Typically, the central portion of the lens (Zone 1) is used to view the fundus up to 30° from the optic nerve/macula. In the illumination ring design of FIG. 27(a), the central axis is left open and unblocked.

Because three-mirror lenses are often used at the slit lamp, power for the illumination ring can typically be supplied via an integrated battery 1438 since the expected treatment time is less than the expected power capacity of around 10-60 min for the battery alone (may be used in overdrive mode). All here presented illumination modules can also be powered via a wired connection e.g. FIGS. 25, 1448 to an external power source 1453. For example in a surgical OR setting under a surgical microscope a wired connection to an operating room external power module can be used, if a long surgery time is expected, for example for a complex vitrectomy/retina surgery.

This illumination ring can be made disposable (especially if the three-mirror lens it attaches to is disposable, as some modern ones are made of PMMA and single-use). Alternatively, a reusable ring can be sterilized and used with a sterilizable lens.

Automated 360-Degree Angle Diagnostics: Referring to FIGS. 26 and 27, the principles of the multi-mirror illumination system can be extended to automated diagnostic grading. In one embodiment, the lens 1410 is a four-mirror gonioscopy lens, and the illumination ring 1430 comprises an array of four miniature cameras 1441, each optically aligned with a respective mirror. This configuration captures a simultaneous 360-degree view of all four quadrants of the anterior chamber angle. A processor analyzes the composite imagery to automatically grade trabecular meshwork pigmentation or detect pathologies. Alternatively, the illumination module 1430 may enclose a motorized internal ring carrying a single camera that rotates 360 degrees around the optical axis to perform a panoramic scan. This automated documentation supports diagnostic reimbursement codes requiring complete angle analysis prior to laser therapy.

Vitreous/Retina Illumination Ring for Retina, Floater, Fundus Visualization and Procedures: Some embodiments can be configured to illuminate the posterior segment of the eye—the vitreous humor and retina—using a contact lens approach. FIG. 19(a) and (b) show typical retina lenses that come in different wide-angle configurations. A version of the illumination ring shown in FIG. 20 is adapted to fit over such retina lenses and provides homogeneous illumination throughout the entire accessible retina area that the lenses from FIG. 19 provide. Furthermore, individual LEDs 1431 on the ring of FIG. 20 are aimed with a light cone of typically 20-90 degrees towards different regions of the retina and can be individually turned on and off using microswitches 1480 or remote control to only bring light to the desired treatment or diagnostic area of the retina if preferred.

During vitreoretinal surgery, surgeons typically rely on a fiber-optic probe inside the eye for illumination. However, an external ring light on the contact lens can supplement or replace this by illuminating the fundus from the outside, improving visibility without extra instruments in the eye. An open-ring design like that of FIG. 20 would not interfere with the surgeon's view or instrument access from the side, but would shine light through the pupil from 360° around its edge. The light sources 1431 are vertically near the pupil (just a few mm above) yet are coming from an open outer ring with a diameter of around 25-50 mm (depending on the retina lens used underneath the light ring of FIG. 20). Because of this geometry, the lights can be angled for each light 1431 such that the light beam goes underneath the iris (using, e.g., a vitreous lens as in FIG. 15) on the opposite side of the pupil. These shallow light beams will illuminate high up in the peripheral retina and therefore can eliminate the need for the use of directly and invasively inserted fiberoptic light probes. This could allow non-invasive visualization of the peripheral retina or vitreous strands and will free up one surgeon's hand, a substantial improvement in performance and safety. In addition, multiple spectral ranges could be used (some ring LEDs could be infrared, for example, to be picked up by a heads-up display system for viewing blood flow or other details).

These retina lenses and illumination rings can also be used for procedures like YAG laser vitreolysis (treatment of floaters) or newer ultrashort photodisruptive pulse laser treatments (femto or pico) or detailed examination of the retina and vitreous without traditional illumination from an invasive fiber light probe, or a typical slit lamp or surgical microscope built-in illumination module.

The ring shown in FIG. 20 carries a set of LEDs (for instance, 4-10 LEDs) around its inner circumference. When used on a retina/vitreous lens (e.g., FIG. 19) these LEDs are angled to send light through the pupil and into the vitreous cavity. Unlike the gonio case, here the light needs to travel farther and illuminate a larger volume (the entire vitreous or a wide field of retina). Thus, higher power LEDs or those with focusing optics might be employed. The beams from the LEDs intersect in the vitreous, creating an illuminated volume wherein floaters (which are tiny opacities in the vitreous gel) will scatter light and become highly visible. Because floaters are more apparent when backlit or when casting shadows on the retina (Flashes and Floaters Charles Retina Institute), providing a bright background illumination in the eye (like a uniformly lit retina) can make them stand out (Flashes and Floaters-Charles Retina Institute). The ring's LEDs effectively turn the retina into a bright backdrop, so the ophthalmologist can easily see the floaters as dark moving specks against a lit field. In a darkened eye, vitreous strands usually have low contrast. But if lit stereoscopically (from various angles), the shadows or reflections of the floaters can become more pronounced, helping the surgeon or an automated system localize them in 3D space. One might blink the LEDs in sequence or vary their intensity to create a sense of depth through motion parallax as well. Once a floater is identified and its distance determined, a focused laser can be applied to evaporate it (laser vitreolysis). The ring provides a wide, encompassing illumination that internal fiber probes cannot, and it does so non-invasively (no need to insert a light into the vitreous).

Furthermore, two outer cameras 1441 with a viewing axis tilted towards the central retina can pinpoint the location of vitreous strands and floaters in 3D when combined with image processing. This 3D image data allows for auto tracking and auto alignment of a photodisruptive pulsed laser beam and thereby creating a go/no-go feedback system for the surgeon on when and where to fire the laser to cut the strands and floaters, or it can enable semiautomatic or fully automatic treatment procedures. Thus, embodiments can advantageously open up new possibilities for diagnostic lighting techniques including 3D visualization and treatment guidance in the vitreous body.

For retinal visualization, the ring can further function like a light source of an indirect ophthalmoscope. Traditional binocular indirect ophthalmoscopy uses an external lamp on the examiner's head; here the lamp is essentially on the patient's eye. The advantage is potentially a wider field of view and less glare from corneal reflections due to the angle of incidence.

Cataract Surgery Capsulotomy Lens Illumination: For cataract surgery, particularly laser-assisted (femtosecond or picosecond) capsulotomy, a contact interface lens (e.g., as in FIG. 15(a) and (b)) is typically placed on the patient's cornea. This lens helps couple the laser into the eye and also often provides visualization of the anterior lens capsule. However, the laser systems (e.g., femtosecond or picosecond lasers) usually have their own integrated imaging (OCT or video) and sometimes ambient light is kept low. Some embodiments can be configured to provide an illumination ring for a capsulotomy lens (for example, a ring as in FIG. 20 adapted to fit over the lenses in FIG. 15) to enhance the surgeon's view of the anterior segment during these critical steps.

This ring (e.g., FIG. 20) is similar in shape to the gonio illumination rings (e.g., FIG. 18) but is in some embodiments specifically designed for a capsulotomy lens which usually has a broader contact surface. The ring holds, for example, 4 to 10 LEDs evenly spaced around its circumference. These LEDs are oriented generally downward and slightly inward, so that their light uniformly washes the anterior chamber with illumination. The goal is homogenous lighting of the iris, anterior lens capsule, and pupil from all sides. This is particularly useful when performing a manual alignment of an anterior laser capsulotomy procedure or for confirmation of capsule tags or checking the completeness of a capsulotomy opening created by the laser. The illumination intensity can be adjustable to remain at a sufficient but comfortable level for the patient.

The ring attaches firmly to the capsulotomy lens (it could clip on or magnetically attach if the lens has a metal band) so that even if the patient's eye moves slightly or if there is surgeon manipulation, it stays in place. It should also detach easily for cleaning or, if it's a single-use component, be cheap enough to dispose.

In another embodiment, the ring of FIG. 20 is configured to illuminate the peripheral iris attached to an iridotomy lens (e.g., FIG. 15(b), (c), or (d)). Another embodiment of the illumination ring in FIG. 20 is configured to provide optimized illumination for multiple procedures in one device. For example, a ring of FIG. 20 mounted on top of lens FIG. 15(b) allows iridotomy and capsulotomy procedure with the same lens, or a ring on lens FIG. 15(d) configured for gonio (angle) treatments and iridotomy with the same lens.

Posterior Capsule (YAG Capsulotomy) Lens Illumination: Another use-case for the illumination ring concept on top of a capsulotomy lens is after cataract surgery, to focus the laser on the posterior capsule to remove opacification. Integrating a light ring (e.g., as in FIG. 20) on such a lens can provide stable illumination of the capsule. Typically, when viewing the posterior capsule and any opacification (fibrosis) on it, the surgeon might struggle with reflections and glare from the slit lamp. A ring light would uniformly illuminate the capsule through the pupil, potentially with cross-polarized light to cut down glare. This standardized lighting can make it easier to see the extent of opacification and ensure the YAG laser can be aimed at the correct spots. Moreover, because a capsulotomy lens must be precisely aligned (centered on the visual axis), an alignment aperture or markings on the ring can assist in maintaining that alignment, similar to how the gonio ring's aperture helps with laser alignment. After use, a disposable capsulotomy lens with an attached illumination ring could simply be discarded, eliminating any risk of contamination from one patient to the next.

Polarization control can be particularly beneficial in these embodiments. It is known that using orthogonally polarized light for illumination and observation can minimize reflections from the cornea and lens surfaces (Preserving polarization maintaining photons for enhanced contrast imaging of the retina-PMC). For example, a polarizing filter can be placed over some or all LEDs (such as those in the embodiments of FIG. 23 and FIG. 25), all aligned to the same polarization axis. The observer (or a camera or microscope) then views through a cross-polarizer (perpendicular axis). Reflected light from the cornea and lens, which maintains the original polarization, will be largely blocked, whereas the light that has traveled into the eye and reflected off the retina or scatter from floaters (which may depolarize or change polarization) can pass through. This technique reduces glare and enhances contrast of retinal details. However, one must consider that strictly orthogonal polarization might also eliminate some desired retinal reflections; as recent research suggests, a combination of parallel and orthogonal polarized light can be used to preserve certain retinal image components while still cutting down glare. In practical terms, the ring (e.g., that of FIG. 20) can offer switchable polarization modes or a removable polarizer that the surgeon can use as needed.

All LED embodiments here described (1414, 1415, 1416, 1431, 1432, 1440, 1449, 1477 and 1478) can employ any wavelength to further optimize the specific illumination properties. Different LED colors on the vitreoretinal ring can dramatically change what structures are emphasized. For instance, green light (around 520-570 nm) enhances the visibility of retinal blood vessels and hemorrhages by providing high contrast (red blood on green background appears very dark) (See, e.g., Red-Free (Green) Filter-Enhanced Gonioscopy with Smartphone: A Pilot Study, 2024, Md Iftekher Iqbal, the entire contents of which is incorporated herein by reference). Ophthalmic examination often uses a green (red-free) filter to highlight the nerve fiber layer and blood vessels (Monochromatic Fundus Photography) (See, e.g., Red-free visualization enhances ease of laser therapy for retinopathy of prematurity, Shakha, 2025, Chandra, Parijat, the entire contents of which is incorporated herein by reference). By incorporating a green LED mode, the ring can allow the examiner to detect subtle nerve fiber layer defects or small diabetic microaneurysms more easily. Blue light (˜450 nm) can help excite fluorescence (for example, fluorescein dye in angiography or the natural fluorescence of the optic disc for nerve assessment) and also increases scatter from small opacities, potentially making tiny floaters or vitreous cells more visible (Rayleigh scatter is stronger for short wavelengths). Blue illumination is also used with fluorescein to check corneal integrity, so if this ring were used in an anterior segment exam, it could highlight corneal staining (though primarily this device is for posterior). Red or infrared light penetrates deeper and can be used to see through mild hemorrhages or cataracts that scatter shorter wavelengths; IR illumination is invisible to the patient (more comfortable) and can be paired with IR-sensitive cameras for imaging the choroid or for alignment purposes. Indeed, as mentioned in the provisional spec, infrared LEDs could be used to reveal details like deep collector channels behind the trabecular meshwork when combined with IR cameras. Similarly, for retina, IR can visualize deeper choroidal structures.

Trabecular meshwork and angle structures: These can be made more visible with certain color tints. A cyan/blue-green illumination emphasizes the pigmented trabecular meshwork by providing a high contrast (pigment absorbs the shorter wavelengths and stands out) (Use of Digital Methods to Optimize Visualization during Surgical Gonioscopy). The referenced MDPI study on digital gonioscopy found that a cyan filter improved trabecular meshwork visibility, and increasing the “color temperature” (adding blue) highlighted Schlemm's canal's red hue. Applying this, our gonio lens illumination ring includes in some embodiments a mode where the LED output is biased towards blue-green to help identify structures like Schlemm's canal (especially if blood is present in it). On the other hand, a magenta/reddish illumination can be used to see blood-filled channels; as they found, highlighting colors close to magenta made blood in Schlemm's canal more apparent (Use of Digital Methods to Optimize Visualization during Surgical Gonioscopy). This can assist surgeons during angle surgeries or angle laser treatments to ensure collector channels are identified by a slight blood reflux which is then lit up by the magenta light. The ring device is in some embodiments designed to cycle through such color modes to allow the surgeon to optimize the view for different targets—a feature particularly useful in combined procedures (e.g., checking both angle and retina).

Vitreous floaters: These tend to be translucent but cast shadows on the retina. Using a bright, broad-field illumination (like the ring provides) makes these shadows more obvious. Additionally, shorter wavelength light (blue) will scatter off small particles in the vitreous more strongly, possibly rendering smaller floaters visible. An example scenario: under normal white light, a patient's tiny vitreous strands are nearly invisible, but under a blue LED illumination from the ring, they appear as shimmering specks due to increased scatter. If the device had a strobe capability, briefly pulsing the light can also enhance detection of motion of floaters relative to static background.

Collector channels and outflow pathways: In some embodiments, the gonio lens or direct angle lens can be used in conjunction with tracer dyes or blood reflux to view aqueous outflow pathways. An LED emitting in the UV or violet range (˜405 nm) could excite fluorescein dye injected in Schlemm's canal, causing it to fluoresce green and reveal the collector channel locations—much like angiography but for the anterior segment. The devices can be configured to accommodate such diagnostic techniques as well (with UV LEDs and the appropriate observation filters).

In some embodiments, polarized light control and multi-wavelength illumination can be incorporated into the device in order to allow tailored lighting for different structures. Some embodiment can comprise one or more of: orthogonal polarizers to cut glare for cornea and iris examinations, parallel polarization to preserve detail when needed (Preserving polarization maintaining photons for enhanced contrast imaging of the retina-PMC); green light for retina and angle pigmentation (Red-Free (Green) Filter-Enhanced Gonioscopy with Smartphone: A Pilot Study, 2024, Md Iftekher Iqbal, the entire contents of which is incorporated herein by reference); blue light for vitreous and cornea (especially with fluorescein); red/IR for deep tissue and patient comfort; or filtered spectrums like cyan or magenta to accentuate certain anatomical or pathological features (Use of Digital Methods to Optimize Visualization during Surgical Gonioscopy). The result can be a highly versatile illumination system that a practitioner can adjust in real-time to get the best possible view of whatever ocular structure is of interest, thereby improving both the diagnostic capability and therapeutic precision.

Mechanically, the vitreoretinal illumination ring can resemble a larger diameter gonio ring. It must accommodate the pupil (typically dilated to ˜7-8 mm for such posterior exams) and the contact lens which often has broad optics. It may have partial sections where no LEDs are present to allow instruments (like a YAG laser probe or vitreous cutter in surgery) to pass if needed—though in laser floater treatment, usually only the YAG laser beam through the pupil is needed. If needed, the ring can be made in sections (e.g., a half-ring that illuminates from the top half only, leaving the bottom half open for instrument access or for the patient's inferior field).

Material Choices and Sterilization: All embodiments described can be manufactured using biocompatible materials commonly used in ophthalmic instruments. For optical elements (like the contact lens that touches the cornea or any window in front of LEDs), PMMA (polymethyl methacrylate) or medical-grade polycarbonate are preferred for their clarity and hardness. The body or housing (ring, cap, lens frame) can be made from plastics like ABS (acrylonitrile butadiene styrene) for disposable versions or PEEK (polyether ether ketone) for high-end reusable versions, as PEEK is highly resistant to chemicals and heat. Some parts could be 3D-printed using biocompatible nylon or photopolymer resins (for rapid prototyping or custom fits). Electrical components (LEDs, batteries, circuits) are enclosed or potted in silicone or epoxy to seal them from fluids and allow safe sterilization.

For reusable devices, sterilization is critical. Preferred methods are low-temperature processes: EtO gas sterilization is effective for delicate electronics and is widely used in hospitals for tools like endoscopes. Hydrogen peroxide plasma (e.g., STERRAD) is another method that operates at low temperature and is compatible with many plastics and electronics. Both these methods penetrate into crevices and eliminate microorganisms without soaking the device (which could corrode circuits) or heating it excessively. Devices can also be designed to be waterproof (IP68 or similar) so that they can withstand immersion in disinfectant solutions or even autoclave to a limited degree. For instance, a PEEK-bodied ring with a glass window could potentially be autoclaved if the battery is removable (batteries generally should not be heated). If using autoclave, one might use medical-grade silicone for the housing which can handle heat and is flexible (though silicone is less durable and could absorb chemicals).

Disposable devices are typically pre-sterilized by the manufacturer. They can be sealed in a sterile pouch and sterilized via gamma irradiation or electron beam, or also EtO which is common for single-use medical plastics. The LEDs and circuits are robust enough to handle those processes.

In terms of assembly, the electronics used (LEDs, microcontroller for PWM, etc.) are low-voltage and can be powered by small batteries safely. All devices are designed to comply with ophthalmic safety standards regarding light exposure. In one version of this disclosure, it is explicitly stated that the illumination intensity and wavelength distributions are within the limits set by ISO 15004-2, ensuring that the patient's retina is not over-exposed to hazardous light levels during use. Likewise, any integrated aiming or treatment lasers used in conjunction with the device would adhere to ANSI Z136.1 classifications for laser safety, and the device's design (e.g., with alignment apertures and beam guards) aids in maintaining those safety margins.

Alternative Embodiments and Variations: While specific embodiments have been described for gonioscopic lenses, three-mirror or 4-mirror lenses, capsulotomy/iridotomy lenses, and vitreoretinal visualization, the concept of integrated illumination can extend to other ophthalmic instruments. For instance, a continuous 360° illumination ring could be adapted for a scleral depressor lens or a gonioscopy surgical lens used in goniotomies (providing light during microinvasive glaucoma surgery). Head-mounted display integration is another possibility: the small cameras on these devices could feed live video to augmented reality glasses for the surgeon. Furthermore, although LEDs are the preferred light source due to their small size and efficiency, future light sources like micro-lasers or OLED light patches could be used in similar ways. Optionally, some embodiments can also incorporate feedback sensors—for example, a light sensor on the device could measure reflected light intensity and automatically adjust LED brightness to optimal levels, or shut off the device after use.

The scope of the present disclosure is intended to include these and other variations that achieve the basic goal of providing improved, localized illumination for eye exams and surgery. The claims that follow define the protected aspects of the various inventions disclosed herein. Any combination of features from different embodiments is contemplated, as long as they are not mutually exclusive. For example, a three-mirror lens illumination ring could also include a central camera; a gonio lens could have both embedded LEDs and an attachable supplemental ring for extra brightness; a capsulotomy lens ring could be made disposable while its controller is reusable, etc. The descriptions herein enable those skilled in the art to make and use the gonio lens device and related components in its various forms.

In all the described embodiments, a key advantage is that the illumination can be brought closer to the target and made to move in unison with the viewing instrument. This fundamentally improves the ergonomics and efficacy of ophthalmic procedures. The surgeon can rely on on-board illumination that remains optimal without further adjustment, which is particularly valuable in complex procedures where the surgeon's attention is at a premium. Patients benefit from shorter procedure times (since less fiddling with equipment is needed) and often from reduced light exposure to sensitive parts of the eye (since light is better directed only where needed). Furthermore, by designing many embodiments to be disposable and battery-powered, issues of sterility and setup are minimized. Each patient can have a fresh illumination device, ensuring consistent performance and eliminating the risk of infection transmission.

Advantages of Disposable Illumination Rings vs. Fixed Light Sources: In accordance with some embodiments, the presently discussed solutions also contrast with the traditional fixed illumination of slit lamps or microscopes in some or all of the following ways:
1. Consistent, High-Quality Illumination: The disposable ring provides a standardized, pre-calibrated lighting environment for each procedure. Because the LEDs have known output and geometry relative to the lens, the illumination on the target is consistent every time. This consistency ensures that laser focusing is accurate—there is no guesswork due to variable reflections or shadows. In fixed systems, the quality and angle of illumination can vary with each setup or if the lamp alignment drifts, which in turn can affect the surgeon's ability to properly focus a laser on a minute target. The ring, being replaced each time, always starts in an optimal state and delivers bright, even lighting at the correct focal plane, thereby enhancing laser focus accuracy.
2. Reduced Surgeon Workload through Pre-alignment: The ring's lights are essentially pre-aligned with the optical path of the lens. When the surgeon attaches the ring to the lens, they do not need to spend time adjusting angles or intensity—the configuration is designed to be optimal by default. In procedures using femtosecond or picosecond lasers (which often involve complex machine setups and timing), having the illumination taken care of is a significant relief. The surgeon can devote attention to controlling the laser parameters and patient eye position, rather than continuously tweaking a slit lamp. The net effect is a more streamlined procedure, where the surgeon's workflow is simplified to: position lens->turn on light->perform treatment. This is especially beneficial during high-precision, time-sensitive laser applications.
3. Optimized for Dual Optical Paths: Modern ophthalmic work often involves dual optical paths—one for visualization (eye or camera) and one for a laser or diagnostic beam. Proprietary microscope systems try to combine these, but introducing external light into such systems can be inefficient due to beam splitters and filters. The illumination ring bypasses these complexities by delivering light directly to the eye, not through the microscope optics at all. This allows the use of customized LEDs (with specific wavelengths or polarization) that might not have been compatible with the microscope's own illumination system. For example, if a proprietary system couldn't easily use UV or IR illumination due to filter coatings, an LED of that wavelength on the ring can be used freely to achieve a desired imaging effect. Thus, the ring complements dual-path setups by providing a parallel, dedicated illumination path that doesn't interfere with or dilute the primary imaging/laser path.
4. Minimized Beam Clipping and Obstruction: As discussed, large aperture laser beams are at risk of being clipped by anything that isn't perfectly aligned. A movable lamp might be moved in front of the microscope/laser focusing lens and thereby clip part of the laser or visualization beam. The disposable ring, on the other hand, is always in front of the lens and is designed with a clear aperture for the beam, minimizing any chance of obstructing the beam. Even the ring's physical profile is made thin to avoid sticking out into the beam's space. In essence, the ring becomes almost a part of the lens, so if the laser can fit through the lens, it will fit through the ring. By eliminating extra fixtures in the vicinity of the beam, the risk of beam interference is greatly reduced.
5. Enhanced Compatibility with Guidance Systems: If the surgical setup includes auto-guidance cameras or tracking systems (for example, an eye tracker that follows the pupil, or a system that detects eye structures to aim the laser), consistent lighting is critical. These systems often rely on machine vision, which can be fooled by glare, shadows, or changes in illumination. The integrated ring provides a stable lighting scenario that can be fine-tuned to the needs of the camera (even using invisible IR light so as not to disturb the surgeon's view). Because the lighting is stable and moves with the eye lens, the tracking system always sees the anatomy under the same lighting conditions, improving its reliability. In contrast, a fixed external light might create reflections that move unpredictably relative to the tracking camera when the gonio lens moves, potentially confusing the system. The ring essentially synchronizes the illumination with the tracking coordinate system, making features easier to detect by automated means.
6. Aligned with Gonioscopic Mirrors vs. Fixed Angles: A slit lamp's illumination usually comes from a fixed angle (e.g., 30 degrees to the left or right of the observation axis). However, a gonioscopic lens mirror might be at 60 degrees inside the eye relative to the observation axis. This mismatch can lead to suboptimal illumination—parts of the mirror's view remain dark or poorly lit. The disposable ring, by virtue of being mounted on the lens, can place its LEDs at angles that directly correspond to the mirror's line of sight. Essentially, the light is delivered from the perspective of the mirror, something a fixed lamp cannot do well. During gonio-laser procedures (like SLT), this means the trabecular meshwork is illuminated along the same axis that the laser will take after reflection, improving visualization and laser coupling. The fixed lamp approach would always be hitting at an off-axis angle relative to the mirror, causing some light loss and uneven lighting of the target. The ring fixes that by making the illumination axis congruent with the viewing/laser axis (through the mirror).
7. Standardized Diagnostic Lighting: Ophthalmologists often compare findings across time (e.g., angle pigmentation from one visit to the next) or between eyes. Using a disposable ring that has known, factory-set illumination properties ensures that each examination is done under the same lighting conditions. Over time, this builds consistency in diagnostic imaging. For instance, if a certain LED brightness and color shows Schwalbe's line best, using the same ring model each time on each patient yields comparable views, rather than relying on manual lamp adjustments that could differ. This standardization can improve the accuracy of glaucoma diagnostics, where subtle changes in the angle may indicate progression. Additionally, teaching and collaboration benefit—if every clinician uses the same illumination ring settings, images and observations are more reproducible across clinics.
8. Sterility and Maneuverability: The disposable nature of the ring means each unit is pre-sterilized and opened fresh for the patient. There is no risk of biological residue from previous use, unlike a slit lamp that could, for example, have contaminants on the lamp housing or mirrors. Moreover, because the ring is battery-powered and cordless, the surgeon has full freedom to maneuver the contact lens on the eye. There are no fiber optic cables taped to the lens or wires running to a power source that might tug or impose drag on the lens as it's rotated. This preserves the natural hand feel of using the lens. It is as if one is using a regular diagnostic lens, but with magically perfect illumination. The surgeon can rotate, tilt, or reposition the lens without any encumbrance. In contrast, if using an external light pipe or fiber tethered to a lens, each movement has resistance or requires managing the cable position. By avoiding that, the ring makes the procedure smoother and potentially safer (no sudden slips due to pulling cables).

In light of these comparisons, the disposable illumination ring of some embodiments can clearly offer numerous advantages in surgical efficiency, illumination quality, safety, and consistency. These improvements are not trivial; in a field where short laser time exposure and micrometers of precision matter, having optimal lighting can significantly influence outcomes.

Generalized Embodiments: It should be understood that the examples given (gonioscopic lens, three-mirror lens, vitreoretinal lens, etc.) are various implementations of the core inventive concept: providing integrated or attachable illumination for ophthalmic contact lenses to achieve automatically aligned, high-quality lighting of intraocular structures. The various embodiments disclosed herein are not limited to any specific type of contact lens or procedure. For instance, one could imagine a specialized scleral depressed contact lens for retinal surgery with built-in lights, or an anterior chamber stability lens (used in certain cataract surgeries) with an illumination ring to better view the capsule, or a user interface contact lens that is fixated to the limbus via a vacuum suction ring. All such variations, where the light source is brought to the level of the lens and moves with it, fall within the scope of the inventions disclosed herein.

The foregoing description of embodiments and their advantages has been presented for purposes of illustration and explanation. It is not intended to be exhaustive or limiting as to the precise forms disclosed. Many modifications and equivalent arrangements will be apparent to those skilled in the art. For example, the shapes and sizes of illumination rings can be adapted to different lens models; the number and type of LEDs can be varied; wired power can be used instead of battery in some cases; the control of the illumination could be manual or automatically linked with a laser system, and so on. All such modifications are intended to be included within the scope of the inventions as defined in the appended claims.

All embodiments disclosed herein are intended to be used in accordance with generally accepted ophthalmic surgical safety standards and laser safety guidelines. The integrated illumination sources are designed and calibrated such that their intensity and wavelength output remain within safe exposure limits for ocular tissues, ensuring that they do not cause damage or undue glare. The device can include built-in attenuators or diffusers if needed to meet specific regulatory requirements for ophthalmic instruments. Additionally, any laser usage in conjunction with these illuminated lenses should follow applicable laser safety protocols (for example, using appropriate wavelength filters, interlocks, and protective eyewear for the surgical team). By adhering to industry standards for eye safety, including standards for optical radiation and biocompatibility of materials, the present disclosure provides devices that not only achieve enhanced performance but also assure patient and user safety. These safety considerations are incorporated into the design—such as using materials that comply with ophthalmic use regulations and electrical components that meet medical device standards—so that the final product can be confidently used in clinical settings without violating ophthalmic or laser safety norms.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

### Clause Set 1: Integrated/Attachable Illumination System

Clause 1. An ophthalmic illumination apparatus, comprising: an ophthalmic lens body configured for direct contact with or close proximity to a patient's eye to facilitate viewing or treatment of an intraocular structure; and at least one miniature illumination source coupled to the ophthalmic lens body and positioned to emit light towards the intraocular structure, wherein the emitted light from the at least one miniature illumination source moves in unison with the ophthalmic lens body as the ophthalmic lens body is moved or reoriented relative to the eye, thereby maintaining automatically aligned illumination of a region of interest within the eye.
Clause 2. An ophthalmic illumination system, comprising: an ophthalmic patient interface device adapted to be placed on or coupled to an eye; an illumination module mechanically attachable to and detachable from the ophthalmic patient interface device, the illumination module comprising a plurality of miniature light-emitting elements arranged to direct light through the ophthalmic patient interface device or alongside the ophthalmic patient interface device towards an intraocular target region when the illumination module is attached; and a power source configured to energize the plurality of miniature light-emitting elements.
Clause 3. A cordless ophthalmic illumination device, comprising: an ophthalmic contact lens configured to interface with a cornea of an eye; a housing coupled to the ophthalmic contact lens; one or more light-emitting diodes (LEDs) disposed within the housing and oriented to illuminate an intraocular target; and a self-contained power source disposed within the housing and electrically coupled to the one or more LEDs, wherein the device is free of external wiring during operation.
Clause 4. A method for illuminating an intraocular structure, the method comprising: placing an ophthalmic lens assembly onto a patient's eye, the ophthalmic lens assembly comprising an ophthalmic lens and at least one integrated or detachably coupled miniature illumination source; activating the at least one miniature illumination source to direct light towards a target intraocular structure viewable through or facilitated by the ophthalmic lens; and maneuvering the ophthalmic lens assembly relative to the eye, wherein the illumination from the at least one miniature illumination source automatically follows the maneuvering to maintain illumination of the target intraocular structure without manual adjustment of an external light source.
Clause 5. A method for performing an ophthalmic procedure, comprising: coupling an illumination module to an ophthalmic contact lens, the illumination module comprising integrated light sources and an integrated battery; positioning the ophthalmic contact lens with the coupled illumination module on a patient's eye; energizing the integrated light sources using the integrated battery to provide localized illumination of an intraocular target; and performing a diagnostic or therapeutic action on the intraocular target while the target is illuminated by the integrated light sources.
Clause 6. The apparatus, system, device, or method of any preceding Clause, wherein the ophthalmic lens body or patient interface device is a gonioscopic lens comprising at least one mirror, and wherein the illumination source(s) are positioned and oriented to illuminate a region of the anterior chamber angle viewable via the at least one mirror.
Clause 7. The apparatus, system, device, or method of any preceding Clause, wherein the illumination source(s) are embedded within the material of the ophthalmic lens body adjacent to an optical element.
Clause 8. The apparatus, system, device, or method of any preceding Clause, wherein the illumination module or housing is a ring-shaped or partially ring-shaped cap configured to snap onto or encircle an upper portion of the ophthalmic lens body or patient interface device.
Clause 9. The apparatus, system, device, or method of any preceding Clause, wherein the illumination module or housing defines a central aperture aligned with an optical pathway of the ophthalmic lens or patient interface device.
Clause 10. The apparatus, system, device, or method of any preceding Clause, wherein the power source is a micro-battery integrated within the illumination module or housing.
Clause 11. The apparatus, system, device, or method of any preceding Clause, wherein the power source is a miniature battery having a thickness between 1 mm and 8 mm and a diameter between 4 mm and 23 mm.
Clause 12. The apparatus, system, device, or method of any preceding Clause, further comprising at least one miniature camera coupled to the ophthalmic lens body or integrated into the illumination module, the camera positioned to capture images of the illuminated intraocular structure.
Clause 13. The apparatus, system, device, or method of Clause 12, wherein the at least one miniature camera comprises two cameras arranged to provide stereoscopic imaging.
Clause 14. The apparatus, system, device, or method of any preceding Clause, wherein the illumination source(s) comprise Light Emitting Diodes (LEDs).
Clause 15. The apparatus, system, device, or method of any preceding Clause, further comprising a controller or control electronics configured to adjust at least one of an intensity, a wavelength, a polarization state, or a timing of the emitted light.
Clause 16. The apparatus, system, device, or method of any preceding Clause, wherein the illumination source(s) are configured to provide illumination of selectable different colors, including at least one of white, green, blue, red, cyan, magenta, or infrared light.
Clause 17. The apparatus, system, device, or method of any preceding Clause, further comprising a polarizer associated with the illumination source(s) to emit polarized light.
Clause 18. The apparatus, system, device, or method of any preceding Clause, wherein the apparatus, system, or device, or components thereof, are configured as a single-use disposable unit.
Clause 19. The apparatus, system, device, or method of any preceding Clause, further comprising an alignment aperture integrated with the ophthalmic lens body, illumination module, or housing, configured to facilitate alignment of a treatment beam.
Clause 20. The apparatus, system, device, or method of any preceding Clause, wherein the ophthalmic lens body or patient interface device is configured for vitreoretinal viewing, and the illumination source(s) are positioned to illuminate the posterior segment of the eye.
Clause 21. The apparatus, system, device, or method of any preceding Clause, wherein the ophthalmic lens body or patient interface device is a capsulotomy lens or an iridotomy lens, and the illumination source(s) are configured to illuminate a lens capsule or an iris.
Clause 22. The apparatus, system, device, or method of any preceding Clause, wherein the ophthalmic lens body or patient interface device is a multi-mirror lens, comprising multiple illumination sources dedicated to respective mirrors.
Clause 23. The apparatus, system, device, or method of any preceding Clause, further comprising a central illumination source positioned to emit light coaxially through a pupil of the eye.
Clause 24. The apparatus, system, device, or method of Clause 23, wherein the central illumination source is activated or configured to induce pupillary constriction, thereby opening an anterior chamber angle view.
Clause 25. The apparatus, system, device, or method of any preceding Clause, further comprising status indicator lights integrated with the apparatus, system, or device.
Clause 26. The apparatus, system, device, or method of any preceding Clause, further comprising a user-operable control element on the apparatus, system, or device for controlling the illumination source(s).
Clause 27. The apparatus, system, device, or method of any preceding Clause, further comprising control electronics integrated within the illumination module or housing for managing power and illumination parameters.
Clause 28. The apparatus, system, device, or method of Clause 27, wherein the control electronics include a wireless communication module.
Clause 29. The method of any of Clauses 4-28, further comprising tailoring a spectrum or intensity of the light to enhance visualization of specific tissues.
Clause 30. The method of Clause 29, wherein tailoring the spectrum comprises selecting green or cyan light to enhance visualization of pigmented trabecular meshwork or blood vessels.
Clause 31. The method of any of Clauses 4-30, further comprising capturing images using an integrated miniature camera and utilizing the captured images for automated guidance of a laser treatment.
Clause 32. The method of any of Clauses 4-31, wherein activating the illumination source(s) includes selecting from multiple available wavelengths or polarization states.
Clause 33. The method of any of Clauses 5-32, further comprising, after performing the action, detaching the illumination module and discarding it.
Clause 34. The method of any of Clauses 5-33, wherein energizing the integrated light sources comprises overdriving the integrated LEDs with a current greater than 20 mA provided by the integrated battery.
Clause 35. The method of any of Clauses 4-34, wherein the illumination remains automatically aligned with a field of view of the ophthalmic lens during the procedure.
Clause 36. The apparatus, system, or device of any of Clauses 1-3, 6-28, or 35, wherein the illumination source(s) or LEDs are overdriven with a current between 25 mA and 100 mA supplied by the power source.

### Clause Set 2: Detachable Self-Contained Illumination Accessory

Clause 1. An ophthalmic illumination accessory for an ophthalmic contact lens, comprising: a housing configured for releasable mechanical attachment to an existing ophthalmic contact lens designed for examining or treating an eye; a plurality of miniature illumination sources disposed within or on the housing, arranged to direct light towards a target region within the eye when the housing is attached to the ophthalmic contact lens; and an integrated power source contained within the housing for energizing the plurality of miniature illumination sources, rendering the accessory self-powered and cordless when attached to the contact lens.
Clause 2. A modular ophthalmic illumination system, comprising: an ophthalmic contact lens; and a detachable illumination cap comprising: a body shaped to fit onto at least a portion of the ophthalmic contact lens; multiple LEDs mounted on the body, oriented to illuminate an intraocular structure viewable through the ophthalmic contact lens; a battery secured within the body of the cap to power the multiple LEDs; and a central aperture in the body aligned with an optical pathway of the ophthalmic contact lens.
Clause 3. A single-use disposable illumination module for an ophthalmic lens, comprising: a polymer body configured to mechanically couple to an upper rim of an ophthalmic lens; at least one LED integrated into the polymer body; control electronics integrated into the polymer body; and a battery integrated into the polymer body and electrically connected to the control electronics and the at least one LED; wherein the module is provided in a sterile package and configured for disposal after a single patient use.
Clause 4. A method of providing illumination for an ophthalmic procedure, comprising: selecting an ophthalmic contact lens appropriate for the procedure; attaching a separate, self-contained illumination module to the selected ophthalmic contact lens, the illumination module comprising its own light sources and integrated power source; placing the ophthalmic contact lens with the attached illumination module on a patient's eye; and activating the light sources in the illumination module to illuminate a target tissue without connecting the illumination module to an external power supply.
Clause 5. A method of manufacturing a self-contained ophthalmic illumination accessory, comprising: molding a housing configured for releasable attachment to an ophthalmic contact lens; integrating a plurality of LEDs into the housing, oriented to direct light toward an optical path of the ophthalmic contact lens; integrating control circuitry into the housing; inserting a battery into a compartment within the housing, the battery electrically coupled to the control circuitry and the plurality of LEDs; and sealing the housing to protect the LEDs, control circuitry, and battery.
Clause 6. The accessory, system, module, or method of any of Clauses 1-5, wherein the housing, body, or module is ring-shaped or partially ring-shaped.
Clause 7. The accessory, system, module, or method of any of Clauses 1-5, further comprising a user-operable switch on the housing, body, or module to control activation, intensity, or mode of the illumination sources or LEDs.
Clause 8. The accessory, system, module, or method of any of Clauses 1-5, further comprising status indicator lights configured to convey information regarding power status, battery level, or procedure status.
Clause 9. The accessory, system, module, or method of any of Clauses 1-5, wherein the illumination sources or LEDs include sources of different colors selectable by a user.
Clause 10. The accessory, system, module, or method of any of Clauses 1-5, further comprising at least one integrated miniature camera disposed within the housing, body, or module.
Clause 11. The accessory, system, module, or method of any of Clauses 1-5, wherein the housing, body, or module defines an alignment aperture configured as a guide for a laser beam.
Clause 12. The accessory, system, module, or method of any of Clauses 1-5, wherein the accessory, module, or illumination cap is designed for single-use and is sterile packaged.
Clause 13. The accessory, system, module, or method of any of Clauses 1-5, wherein the illumination sources or LEDs are oriented to provide multi-directional illumination to reduce shadows.
Clause 14. The accessory, system, module, or method of any of Clauses 1-5, wherein the illumination cap, module, or accessory includes control circuitry or control electronics for controlling the LEDs or illumination sources.
Clause 15. The accessory, system, module, or method of Clause 14, wherein the control circuitry or electronics include a microcontroller unit configured to provide independent brightness control, pulsed illumination control, or wavelength switching.
Clause 16. The accessory, system, module, or method of any of Clauses 1-5, wherein the housing, body, or module is adapted for attachment to a gonioscopy lens, and the illumination sources or LEDs are directed towards a mirror of the gonioscopy lens.
Clause 17. The accessory, system, module, or method of Clause 16, wherein the illumination sources or LEDs are disposed within angled channels to precisely target the mirror.
Clause 18. The accessory, system, module, or method of any of Clauses 1-5, wherein the housing, body, or module is adapted for attachment to a three-mirror lens, and the illumination sources or LEDs are positioned to illuminate sectors corresponding to each mirror.
Clause 19. The accessory, system, module, or method of Clause 18, wherein the housing, body, or module includes inward protrusions positioned to reside between mirrors of the three-mirror lens, with illumination sources or LEDs housed in the protrusions and aimed at adjacent mirrors.
Clause 20. The accessory, system, module, or method of any of Clauses 1-5, wherein the housing, body, or module comprises an open region allowing direct instrument access or viewing through the contact lens, alongside illuminated regions.
Clause 21. The accessory, system, module, or method of any of Clauses 1-5, wherein the integrated power source or battery is a coin-cell battery.
Clause 22. The accessory, system, module, or method of Clause 21, wherein the battery is oriented horizontally within the housing, body, or module to reduce an overall height.
Clause 23. The accessory, system, module, or method of Clause 21, wherein the battery is oriented vertically within the housing, body, or module.
Clause 24. The accessory, system, module, or method of any of Clauses 1-5, wherein the integrated power source or battery is configured to overdrive the illumination sources or LEDs with a current greater than 20 mA.
Clause 25. The accessory, system, module, or method of any of Clauses 1-5, wherein the housing, body, or module is constructed from a biocompatible polymer selected from the group consisting of: PMMA, polycarbonate, ABS, PEEK, and nylon.
Clause 26. The accessory, system, module, or method of any of Clauses 1-5, wherein the control electronics comprise a microcontroller and a wireless communication interface.
Clause 27. The accessory, system, module, or method of any of Clauses 1-5, wherein the battery is a Li-Ion battery intentionally overdriven to achieve a current output of 25 mA to 100 mA for a duration of 5 to 60 minutes.
Clause 28. The method of any of Clauses 4-27, further comprising detaching the illumination module from the ophthalmic contact lens after the procedure and discarding the illumination module.
Clause 29. The method of any of Clauses 4-28, wherein activating the light sources includes providing polarized light.
Clause 30. The method of any of Clauses 4-29, further comprising adjusting the illumination from the module to visualize different depths within the vitreous cavity.
Clause 31. The method of any of Clauses 4-30, wherein the illumination module is attached to a capsulotomy lens, and the light sources illuminate an anterior or posterior lens capsule.
Clause 32. The method of any of Clauses 5-31, further comprising sterilizing the sealed housing using gamma irradiation, electron beam, or ethylene oxide gas.
Clause 33. The method of any of Clauses 5-32, wherein integrating the plurality of LEDs comprises positioning the LEDs within angled recesses in the housing.
Clause 34. The method of any of Clauses 5-33, wherein the control circuitry is configured to overdrive the LEDs beyond their standard operating current specification.
Clause 35. The accessory, system, module, or method of any of Clauses 1-5, further comprising an RFID tag embedded in the housing, body, or module.
Clause 36. The accessory, system, module, or method of any of Clauses 1-5, further comprising a pressure sensor integrated into the housing, body, or module to sense pressure against the eye.
Clause 37. The accessory, system, module, or method of any of Clauses 1-5, wherein the illumination cap or module is configured to attach to the ophthalmic contact lens via a snap fit, friction fit, or magnetic attachment.
Clause 38. The accessory, system, module, or method of any of Clauses 1-5, wherein the housing, body, or module includes a grooved outer surface for enhanced grip.
Clause 39. The accessory, system, module, or method of any of Clauses 1-5, wherein the housing, body, or module is adjustable to fit ophthalmic contact lenses of different diameters.

### Clause Set 3: Integrated Imaging and Guidance System

Clause 1. An ophthalmic diagnostic and therapeutic device, comprising: a body adapted for placement on or near an eye, the body defining an optical path for viewing or treating an intraocular structure; at least one illumination source integrated within or fixedly attached to the body, positioned to illuminate the intraocular structure; and at least one miniature imaging sensor integrated within or fixedly attached to the body, positioned to capture images of the illuminated intraocular structure, wherein the illumination source and the imaging sensor share a substantially common field of view and move in unison with the body.
Clause 2. An ophthalmic imaging system, comprising: an ophthalmic lens assembly configured for contact with a patient's eye; a plurality of light-emitting diodes (LEDs) integrated with the lens assembly to illuminate a target area within the eye; one or more miniature cameras co-located with the LEDs on the lens assembly, oriented to image the target area illuminated by the LEDs; and a data transmission interface operatively connected to the one or more miniature cameras for outputting image data.
Clause 3. A stereoscopic ophthalmic visualization device, comprising: an ophthalmic contact lens; an attachment coupled to the ophthalmic contact lens; at least one illumination source integrated into the attachment; and two miniature cameras integrated into the attachment, the cameras being spatially separated and oriented to capture stereoscopic images of an intraocular structure illuminated by the at least one illumination source.
Clause 4. A method for enhanced ophthalmic visualization and guidance, comprising: positioning an ophthalmic device on a patient's eye, the device comprising integrated illumination sources and integrated imaging sensors; activating the illumination sources to illuminate an intraocular target; simultaneously capturing real-time images of the illuminated intraocular target using the imaging sensors; and utilizing the captured real-time images for at least one of guiding a therapeutic procedure, documenting the procedure, or providing an enhanced visual display to an operator.
Clause 5. A method for 3D localization of intraocular structures, comprising: placing an ophthalmic lens assembly on an eye, the assembly comprising integrated illumination and two spatially separated integrated cameras; illuminating an intraocular structure using the integrated illumination; capturing stereoscopic images of the intraocular structure using the two integrated cameras; and processing the stereoscopic images to determine a 3D location of the intraocular structure relative to the ophthalmic lens assembly.
Clause 6. The device, system, or method of any of Clauses 1-5, wherein the body or lens assembly is an ophthalmic contact lens.
Clause 7. The device, system, or method of any of Clauses 1-5, wherein the body, attachment, or illumination sources and imaging sensors/cameras are integrated within a detachable ring or cap configured to mount onto an ophthalmic contact lens.
Clause 8. The device, system, or method of any of Clauses 1-5, wherein the device, system, or lens assembly is a gonioscope comprising a mirror, and the illumination sources/LEDs and imaging sensors/cameras are positioned to illuminate and image the anterior chamber angle via the mirror.
Clause 9. The device, system, or method of any of Clauses 1-5, wherein the two miniature cameras are positioned on opposite sides of a mirror with viewing angles tilted towards the mirror.
Clause 10. The device, system, or method of any of Clauses 1-5, further comprising a processor configured to analyze the image data or stereoscopic images from the imaging sensors or cameras to provide guidance for laser alignment.
Clause 11. The device, system, or method of Clause 10, wherein the guidance includes detecting anatomical landmarks and automatically adjusting a laser position or focus.
Clause 12. The device, system, or method of any of Clauses 1-5, wherein the device or system comprises at least two imaging sensors or cameras arranged to provide stereoscopic or 3D imaging.
Clause 13. The device, system, or method of any of Clauses 1-5, further comprising an integrated power source for the illumination sources/LEDs and the imaging sensors/cameras, rendering the device cordless.
Clause 14. The device, system, or method of any of Clauses 1-5, wherein the data transmission interface is a wireless interface integrated into the lens assembly or attachment.
Clause 15. The device, system, or method of any of Clauses 1-5, wherein the data transmission interface comprises a wired connection to an external computer or electronics module.
Clause 16. The device, system, or method of any of Clauses 1-5, wherein the illumination sources/LEDs are adjustable in wavelength or intensity based on feedback from the imaging sensor/camera or operator input.
Clause 17. The device, system, or method of any of Clauses 1-5, wherein the illumination sources/LEDs are configured to emit infrared (IR) light and the imaging sensors/cameras are IR-sensitive.
Clause 18. The device, system, or method of Clause 17, wherein the IR light and IR-sensitive cameras are used to visualize structures behind the iris or within the scleral wall.
Clause 19. The device, system, or method of any of Clauses 1-5, further comprising an alignment aperture, wherein the imaging sensor or camera is configured to monitor a laser aiming beam passing through the alignment aperture.
Clause 20. The device, system, or method of any of Clauses 1-5, wherein the lens assembly or device is configured for vitreoretinal visualization, the illumination sources/LEDs provide wide-field illumination, and the cameras capture movement of vitreous floaters.
Clause 21. The device, system, or method of any of Clauses 1-5, wherein the device is configured for visualizing vitreous floaters, and stereoscopic images are used to generate 3D localization data for automated laser tracking and alignment.
Clause 22. The device, system, or method of any of Clauses 1-5, wherein the imaging sensor or camera provides data for tracking eye movement.
Clause 23. The device, system, or method of any of Clauses 1-5, wherein the device, system, or lens assembly and integrated components are configured as a single-use disposable unit.
Clause 24. The device, system, or method of any of Clauses 1-5, wherein the at least one miniature imaging sensor or camera has a diameter between 0.3 mm and 8 mm.
Clause 25. The device, system, or method of Clause 24, wherein the diameter is between 1 mm and 3 mm.
Clause 26. The device, system, or method of any of Clauses 1-5, wherein the device includes the illumination source, the cameras, a battery, and wireless transmission electronics integrated into a detachable ring module.
Clause 27. The method of any of Clauses 4-26, wherein utilizing the captured real-time images includes displaying them on a heads-up display or monitor for the operator.
Clause 28. The method of any of Clauses 4-27, further comprising processing the captured images to detect specific anatomical landmarks, such as the trabecular meshwork or Schwalbe's line.
Clause 29. The method of any of Clauses 4-28, further comprising recording the captured images and illumination parameters as a record of the procedure.
Clause 30. The method of any of Clauses 4-29, wherein guiding the therapeutic procedure comprises automatically adjusting a focus plane of a therapeutic laser based on the captured images.
Clause 31. The method of any of Clauses 5-30, wherein the intraocular structure is a vitreous floater or strand.
Clause 32. The method of Clause 31, further comprising using the determined 3D location to generate a go/no-go feedback signal for firing a photodisruptive laser at the vitreous floater or strand.
Clause 33. The method of any of Clauses 5-30, wherein the intraocular structure is the anterior chamber angle, and the 3D location is used to determine a momentary z-focus alignment of a laser focus.
Clause 34. The method of any of Clauses 4-33, wherein the integrated illumination provides a stable lighting scenario synchronized with a coordinate system of the imaging sensors, improving reliability of automated image analysis.
Clause 35. The device, system, or method of any of Clauses 1-5, further comprising a central imaging sensor positioned to capture a direct axial view through a pupil.
Clause 36. The device, system, or method of Clause 35, wherein the central imaging sensor is used to monitor pupil size or align the device centrally over the pupil.
Clause 37. The device, system, or method of any of Clauses 1-5, wherein the one or more cameras are configured to visualize wavelengths beyond human vision.
Clause 38. The method of any of Clauses 4-37, wherein activating the illumination sources comprises using UV or violet light to excite tracer dyes, and capturing images comprises capturing fluorescence from the excited tracer dyes.
Clause 39. The device, system, or method of any of Clauses 1-5, wherein the body or attachment includes control electronics configured to receive external control signals for the imaging sensor or camera.
Clause 40. The device, system, or method of any of Clauses 1-5, wherein the illumination source and imaging sensor are sealed to withstand sterilization by ethylene oxide gas or hydrogen peroxide plasma.

### Clause Set 4: Advanced Illumination Modalities and Control

Clause 1. An ophthalmic illumination device, comprising: an ophthalmic lens element for placement on an eye; a plurality of illumination sources coupled to the lens element, the plurality of illumination sources including: at least a first set of illumination sources configured to emit light of a first wavelength spectrum; and at least a second set of illumination sources configured to emit light of a second, different wavelength spectrum; and a selection mechanism for selectively activating the first set, the second set, or a combination thereof to provide tailored illumination of an intraocular structure.
Clause 2. An ophthalmic illumination system, comprising: a patient interface device for an eye procedure; one or more light sources coupled to the patient interface device; at least one polarizing element positioned in an optical path of light emitted from the one or more light sources, configured to polarize the light illuminating an intraocular target; and optionally, a complementary polarization analyzer in a viewing path.
Clause 3. A microcontroller-controlled ophthalmic illumination apparatus, comprising: a housing configured to couple to an ophthalmic contact lens; a plurality of LEDs disposed in the housing; an integrated power source; and a control electronics module comprising a microcontroller unit (MCU) electrically coupled to the plurality of LEDs and the integrated power source, wherein the MCU is configured to control the plurality of LEDs according to one or more pre-programmed modes.
Clause 4. A method for optimizing visualization of an intraocular structure, comprising: providing an ophthalmic device in contact with or near an eye, the device having a plurality of integrated or attached light sources capable of emitting light with different characteristics; selecting a specific illumination characteristic from the group consisting of: a specific wavelength, a combination of wavelengths, a polarization state, a multi-directional illumination pattern, and a pulsed illumination sequence; and activating the light sources to illuminate the intraocular structure with the selected specific illumination characteristic to enhance its visibility or the visibility of a specific feature thereof.
Clause 5. A method for controlling an ophthalmic illumination device, comprising: providing an illumination device coupled to an ophthalmic lens, the illumination device comprising multiple LEDs and an integrated microcontroller; receiving a control input, either via a user interface on the device or wirelessly; and in response to the control input, adjusting, via the microcontroller, an illumination pattern of the multiple LEDs by modulating intensity, timing, or wavelength of individual LEDs.
Clause 6. The device, system, apparatus, or method of any of Clauses 1-5, wherein the first wavelength spectrum is visible light and the second wavelength spectrum is infrared light.
Clause 7. The device, system, apparatus, or method of any of Clauses 1-5, wherein the illumination sources or LEDs comprise a set emitting green light (520-570 nm) and a set emitting blue light (˜450 nm) or red light.
Clause 8. The device, system, apparatus, or method of any of Clauses 1-5, wherein the polarizing element is a linear polarizer in front of each light source or LED.
Clause 9. The device, system, apparatus, or method of any of Clauses 1-5, configured for cross-polarization wherein the emitted light is cross-polarized relative to viewing optics to reduce glare from ocular surfaces.
Clause 10. The device, system, apparatus, or method of any of Clauses 1-5, comprising illumination sources or LEDs positioned to provide multi-directional illumination to the same target region simultaneously, reducing shadows.
Clause 11. The device, system, apparatus, or method of Clause 10, wherein the multi-directional illumination involves activating LEDs on an overhead ring and LEDs embedded in a sidewall of a lens.
Clause 12. The device, system, apparatus, or method of any of Clauses 1-5, wherein the lens element or patient interface device is a gonioscopic lens, and the illumination sources or LEDs are oriented relative to a mirror of the lens.
Clause 13. The device, system, apparatus, or method of any of Clauses 1-5, further comprising an integrated camera configured to view the structure under the tailored illumination or polarized illumination.
Clause 14. The device, system, apparatus, or method of any of Clauses 1-5, further including a central illumination source aimed coaxially through a pupil.
Clause 15. The device, system, apparatus, or method of any of Clauses 1-5, wherein the device, system, or apparatus is configured as a detachable ring or cap.
Clause 16. The device, system, apparatus, or method of any of Clauses 1-5, including UV or violet (˜405 nm) LEDs for exciting tracer dyes.
Clause 17. The device, system, apparatus, or method of any of Clauses 1-5, wherein a polarization state is switchable or the polarizing element is removable.
Clause 18. The device, system, apparatus, or method of any of Clauses 1-5, wherein the device, system, or apparatus is disposable.
Clause 19. The apparatus or method of any of Clauses 3-18, wherein the pre-programmed modes include independent brightness control for individual LEDs using Pulse Width Modulation (PWM).
Clause 20. The apparatus or method of any of Clauses 3-19, wherein the pre-programmed modes include pulsed or modulated illumination control.
Clause 21. The apparatus or method of any of Clauses 3-20, wherein the pre-programmed modes include sequential activation of the plurality of LEDs.
Clause 22. The apparatus or method of any of Clauses 3-21, wherein the MCU is configured to monitor battery level and provide a low battery indication or automatic cutoff.
Clause 23. The apparatus or method of any of Clauses 3-22, wherein the control electronics module further comprises a wireless communication interface (e.g., Bluetooth Low Energy, Wi-Fi) configured to receive control signals from an external device or transmit data.
Clause 24. The apparatus or method of any of Clauses 3-23, further comprising an integrated sensor selected from the group consisting of: a pressure sensor, a temperature sensor, and a photodiode, wherein the MCU is configured to process data from the integrated sensor.
Clause 25. The apparatus or method of Clause 24, wherein the MCU is configured to provide visual or audio feedback based on the processed data from the integrated sensor.
Clause 26. The apparatus or method of any of Clauses 3-25, further comprising an RFID or NFC tag integrated with the control electronics module.
Clause 27. The method of any of Clauses 4-26, wherein selecting a specific wavelength includes choosing cyan/blue-green illumination to enhance pigmented trabecular meshwork.
Clause 28. The method of any of Clauses 4-27, wherein selecting a specific wavelength includes choosing magenta/reddish illumination to view blood-filled channels such as Schlemm's canal.
Clause 29. The method of any of Clauses 4-28, wherein selecting a specific illumination characteristic includes using blue light to enhance visibility of vitreous floaters by increased scattering.
Clause 30. The method of any of Clauses 4-29, comprising switching between different illumination characteristics during a single procedure.
Clause 31. The method of any of Clauses 4-30, wherein the multi-directional illumination pattern involves activating LEDs on an overhead ring and LEDs embedded in a sidewall of a lens.
Clause 32. The method of any of Clauses 5-31, wherein adjusting the illumination pattern comprises blinking the LEDs in sequence to create a sense of depth through motion parallax.
Clause 33. The method of any of Clauses 5-32, wherein the control input is received wirelessly from an external computer or remote control unit.
Clause 34. The method of any of Clauses 5-33, further comprising monitoring sensor data via the microcontroller and adjusting the illumination pattern based on the sensor data.
Clause 35. The device, system, apparatus, or method of any of Clauses 1-5, wherein the selection mechanism is a microswitch or button on the device.
Clause 36. The device, system, apparatus, or method of any of Clauses 1-5, wherein the illumination sources comprise Vertical-Cavity Surface-Emitting Lasers (VCSELs) configured to provide structured illumination.
Clause 37. The device, system, apparatus, or method of any of Clauses 1-5, wherein the illumination sources comprise OLED light patches.
Clause 38. The method of any of Clauses 4-37, wherein activating the light sources comprises using green light to detect nerve fiber layer defects or microaneurysms.
Clause 39. The method of any of Clauses 4-38, wherein activating the light sources comprises using red or infrared light to visualize through mild hemorrhages or cataracts.
Clause 40. The apparatus or method of any of Clauses 3-39, further comprising a light sensor configured to measure reflected light intensity, wherein the MCU automatically adjusts LED brightness based on the measured intensity.

### Clause Set 5: Alignment and Safety Features

Clause 1. An ophthalmic device for guiding laser procedures, comprising: a body adapted for placement on a patient's eye, the body including an ophthalmic lens; an illumination system integrated with or attached to the body for illuminating a target site within the eye; and an alignment aperture physically defined by the body or an attachment thereto, the alignment aperture being positioned in a path of a therapeutic laser beam and configured to provide visual or sensory feedback regarding centering of the laser beam relative to the aperture before the laser beam reaches the target site.
Clause 2. An ophthalmic illumination and safety system, comprising: an ophthalmic contact lens having an optical axis; an illumination module coupled to the contact lens, the illumination module comprising: light sources for illuminating an intraocular structure; and a physical guide structure defining a clear passage aligned with the optical axis of the contact lens, the guide structure configured such that impingement of an off-axis laser beam on an edge of the guide structure causes a detectable change, thereby indicating misalignment.
Clause 3. An ophthalmic interface with beam clipping detection, comprising: an ophthalmic lens; a housing coupled to the ophthalmic lens, the housing defining an aperture aligned with an optical path of the lens; and at least one sensor positioned adjacent to an edge of the aperture, the sensor configured to detect impingement of a laser beam on the edge of the aperture; and an indicator coupled to the sensor to signal the detected impingement.
Clause 4. A method for ensuring alignment during an ophthalmic laser procedure, comprising: placing an ophthalmic lens assembly on an eye, the assembly including an illumination source and an alignment feature defining an aperture for a laser beam; directing an aiming beam of a laser through the aperture towards a target intraocular structure; observing an interaction of the aiming beam with the alignment feature; and adjusting a position of the ophthalmic lens assembly or the laser until the aiming beam passes cleanly through the aperture without substantial interaction with an edge of the alignment feature, prior to firing a therapeutic laser beam.
Clause 5. A method for detecting laser beam clipping, comprising: providing an ophthalmic interface comprising a lens and a housing defining an aperture for a laser beam path; positioning at least one sensor adjacent to an edge of the aperture; monitoring an output of the sensor while a laser beam is directed toward the aperture; and generating an alert signal if the sensor output indicates impingement of the laser beam on the edge of the aperture.
Clause 6. The device, system, interface, or method of any of Clauses 1-5, wherein the alignment aperture or aperture is part of a detachable cap or module mounted on the ophthalmic lens.
Clause 7. The device, system, interface, or method of any of Clauses 1-5, further comprising a sensor or photodiode at an edge of the alignment aperture or aperture to detect laser light contact or impingement.
Clause 8. The device, system, interface, or method of any of Clauses 1-5, wherein the feedback or detectable change is scattered light visible to an operator resulting from impingement on the edge.
Clause 9. The device, system, interface, or method of any of Clauses 1-5, wherein the illumination module or system further comprises status indicator LEDs, and wherein one of the status indicator LEDs is triggered by detection of laser beam impingement or contact.
Clause 10. The device, system, interface, or method of any of Clauses 1-5, wherein the ophthalmic lens is a gonioscopic lens, and the alignment aperture or aperture is aligned with a mirror of the gonioscopic lens.
Clause 11. The device, system, interface, or method of any of Clauses 1-5, wherein the alignment aperture or aperture has a contrasting ring or coloration to enhance visual detection of beam clipping.
Clause 12. The device, system, interface, or method of any of Clauses 1-5, wherein the illumination system or light sources provide illumination for visualizing the target intraocular structure simultaneously with alignment monitoring.
Clause 13. The device, system, interface, or method of any of Clauses 1-5, wherein the body or guide structure is configured to ensure that if the laser beam fits through the ophthalmic lens, it will fit through the alignment aperture or passage when aligned.
Clause 14. The device, system, interface, or method of any of Clauses 1-5, wherein the physical guide structure or housing is part of a disposable ring.
Clause 15. The device, system, interface, or method of any of Clauses 1-5, designed for use with large-diameter or high numerical aperture laser beams, such as femtosecond or picosecond laser beams.
Clause 16. The device, system, interface, or method of any of Clauses 1-5, wherein the illumination system provides illumination to the target site from a perspective congruent with the viewing/laser axis through the alignment aperture or passage.
Clause 17. The device, system, interface, or method of any of Clauses 1-5, comprising materials compliant with ISO 15004-2 and ANSI Z136.1.
Clause 18. The device, system, interface, or method of any of Clauses 1-5, wherein the sensor is a photodiode.
Clause 19. The device, system, interface, or method of any of Clauses 1-5, wherein the indicator or alert signal triggers a safety interlock to prevent firing of the laser beam.
Clause 20. The device, system, interface, or method of any of Clauses 1-5, further comprising a wireless transmitter configured to send the alert signal to an external laser control system.
Clause 21. The device, system, interface, or method of any of Clauses 1-5, wherein the aiming beam is intentionally made larger than the therapeutic laser beam at the aperture to enhance sensitivity to misalignment.
Clause 22. The method of any of Clauses 4-21, wherein observing the interaction includes visually inspecting for clipping or scattering of the aiming beam at the edge of the alignment feature.
Clause 23. The method of any of Clauses 4-22, further comprising receiving an electronic alert if the aiming beam interacts with the edge of the alignment feature.
Clause 24. The method of any of Clauses 4-23, wherein the ophthalmic lens assembly provides built-in illumination that moves with the lens, maintaining visibility of the target during alignment adjustments.
Clause 25. The method of any of Clauses 4-24, further comprising confirming an armed status of the laser via an indicator on the ophthalmic lens assembly.
Clause 26. The method of any of Clauses 4-25, wherein the illumination source provides polarized light to reduce glare during visualization of the alignment.
Clause 27. The method of any of Clauses 5-26, wherein generating the alert signal includes activating a visual indicator on the ophthalmic interface.
Clause 28. The method of any of Clauses 5-27, wherein generating the alert signal includes sending a signal to a laser control system to disable the laser.
Clause 29. The method of any of Clauses 4-28, wherein the aiming beam is a red alignment laser beam.
Clause 30. The device, system, interface, or method of any of Clauses 1-5, wherein the alignment aperture is defined by a transparent window with opaque borders.
Clause 31. The device, system, interface, or method of any of Clauses 1-5, wherein the illumination system is configured to illuminate the alignment aperture itself to enhance visibility of the edges.
Clause 32. The method of any of Clauses 4-31, wherein the procedure is a photodisruptive laser treatment of the Trabecular Meshwork or a laser iridotomy.
Clause 33. The device, system, interface, or method of any of Clauses 1-5, wherein the illumination system provides standardized lighting conditions to improve consistency of alignment visualization.
Clause 34. The device, system, interface, or method of any of Clauses 1-5, wherein the device is cordless and battery-powered, enhancing maneuverability during the alignment process.
Clause 35. The device, system, interface, or method of any of Clauses 1-5, further comprising markings or etched patterns on the body or module to serve as orientation guides relative to the alignment aperture.

### Clause Set 6: Posterior Segment Illumination System

Clause 1. An ophthalmic illumination device specifically for vitreoretinal procedures, comprising: a contact lens body adapted for wide-field viewing of a posterior segment of an eye, including a vitreous cavity and a retina; and a plurality of miniature light sources coupled to the contact lens body, the light sources being angled and positioned to project light through a pupil of the eye to provide panoramic or targeted illumination within the vitreous cavity and onto the retina, the light sources moving in unison with the contact lens body.
Clause 2. An illumination ring for a vitreoretinal lens, comprising: a ring-shaped body configured for attachment to a wide-field vitreoretinal contact lens; multiple LEDs spaced around the ring-shaped body, oriented to direct light through a pupil of an eye when the lens with the attached ring is placed on the eye; and control electronics configured to individually address and control the multiple LEDs.
Clause 3. An ophthalmic system for visualizing vitreous opacities, comprising: a wide-field contact lens; an illumination module coupled to the contact lens, the module comprising a plurality of LEDs configured to illuminate the vitreous cavity such that vitreous opacities scatter light or cast shadows on the retina; and at least two miniature cameras integrated into the illumination module or coupled to the contact lens, the cameras configured to capture stereoscopic images of the vitreous opacities.
Clause 4. A method for visualizing vitreous floaters for treatment, comprising: placing a wide-field contact lens assembly on a patient's eye, the assembly including a plurality of integrated or attached light sources; activating the light sources to provide broad-field, multi-angle, or sequential illumination of the vitreous cavity, thereby enhancing the contrast or 3D localization of vitreous opacities against an illuminated retinal backdrop or by scattered light; and identifying and targeting said vitreous opacities for laser vitreolysis based on the enhanced visualization.
Clause 5. A method for non-invasive illumination of the peripheral retina, comprising: attaching an illumination ring to a vitreoretinal contact lens, the ring comprising multiple LEDs and having an outer diameter of 25-50 mm; positioning the assembly on an eye; and activating the LEDs such that light beams from the LEDs are directed at shallow angles configured to pass underneath the iris on an opposite side of the pupil to illuminate the peripheral retina without using an invasive fiberoptic light probe.
Clause 6. The device, ring, system, or method of any of Clauses 1-5, wherein the light sources or LEDs include focusing optics to project light deeper into the vitreous cavity.
Clause 7. The device, ring, system, or method of any of Clauses 1-5, further comprising integrated cameras tilted towards a central retina for 3D localization of vitreous strands or floaters.
Clause 8. The device, ring, system, or method of any of Clauses 1-5, wherein the light sources or LEDs are adjustable in intensity.
Clause 9. The device, ring, system, or method of any of Clauses 1-5, wherein the ring or module is configured to attach firmly to the contact lens to maintain position during surgeon manipulation.
Clause 10. The device, ring, system, or method of any of Clauses 1-5, wherein different LEDs or light sources can be modulated in intensity or focus to create a three-dimensional lighting effect.
Clause 11. The device, ring, system, or method of any of Clauses 1-5, wherein the light sources or LEDs are configured to provide blue light (˜450 nm) to increase scattering from small floaters.
Clause 12. The device, ring, system, or method of any of Clauses 1-5, wherein the light sources or LEDs are arranged in an open-ring design or segmented configuration to allow instrument access while illuminating the fundus.
Clause 13. The device, ring, system, or method of any of Clauses 1-5, wherein the device, ring, or module is disposable and designed for single use.
Clause 14. The device, ring, system, or method of any of Clauses 1-5, wherein the light sources or LEDs are configured to illuminate a peripheral retina without requiring an internal fiber optic probe.
Clause 15. The device, ring, system, or method of any of Clauses 1-5, powered by a self-contained battery or an external power source via a wired connection.
Clause 16. The device, ring, system, or method of any of Clauses 1-5, wherein the control electronics are configured to blink the LEDs in sequence.
Clause 17. The device, ring, system, or method of any of Clauses 1-5, wherein the beams from the LEDs or light sources intersect within the vitreous cavity.
Clause 18. The device, ring, system, or method of any of Clauses 1-5, further comprising a processor configured to receive the stereoscopic images and determine a 3D location of the vitreous opacities.
Clause 19. The device, ring, system, or method of Clause 18, wherein the processor is configured to provide auto-tracking and auto-alignment data for a photodisruptive pulsed laser beam.
Clause 20. The device, ring, system, or method of any of Clauses 1-5, wherein the illumination functions similarly to an indirect ophthalmoscope light source, but integrated onto the contact lens.
Clause 21. The device, ring, system, or method of any of Clauses 1-5, wherein the light sources include green light (520-570 nm) LEDs to enhance visibility of retinal blood vessels and hemorrhages.
Clause 22. The device, ring, system, or method of any of Clauses 1-5, wherein the light sources include red or infrared LEDs for visualizing through mild hemorrhages or cataracts or imaging the choroid.
Clause 23. The device, ring, system, or method of any of Clauses 1-5, wherein the illumination ring has an outer diameter between 25 mm and 50 mm.
Clause 24. The device, ring, system, or method of any of Clauses 1-5, wherein the light sources or LEDs have a light cone of 20-90 degrees.
Clause 25. The method of any of Clauses 4-24, wherein activating the light sources includes blinking LEDs in sequence or varying their intensity to create motion parallax for depth perception of floaters.
Clause 26. The method of any of Clauses 4-25, further comprising using two cameras integrated with the contact lens assembly to pinpoint a location of the vitreous opacities in 3D for guiding a laser.
Clause 27. The method of any of Clauses 4-26, comprising using the enhanced visualization to guide a YAG laser, femtosecond laser, or picosecond laser for vitreolysis.
Clause 28. The method of any of Clauses 4-27, further comprising using polarized light from the light sources to reduce glare during visualization.
Clause 29. The method of any of Clauses 5-28, wherein the method frees up one surgeon's hand by eliminating the need for a handheld illumination probe.
Clause 30. The method of any of Clauses 4-29, wherein activating the light sources creates a bright retinal backdrop, making floaters visible as dark moving specks against the lit field.
Clause 31. The device, ring, system, or method of any of Clauses 1-5, adapted for use with a capsulotomy lens to illuminate an anterior or posterior lens capsule.
Clause 32. The device, ring, system, or method of Clause 31, configured for use during laser-assisted capsulotomy to enhance viewing of an anterior lens capsule or to check the completeness of a capsulotomy opening.
Clause 33. The device, ring, system, or method of Clause 31, configured for illuminating a posterior capsule for YAG capsulotomy post-cataract surgery.
Clause 34. The device, ring, system, or method of any of Clauses 1-5, adapted for use with an iridotomy lens to illuminate the peripheral iris.
Clause 35. The device, ring, system, or method of any of Clauses 1-5, adapted for use with a combination lens configured for multiple procedures such as iridotomy and capsulotomy, or gonioscopy and iridotomy.

### Clause Set 7: Power Management and High-Brightness Single-Use

Clause 1. A self-contained ophthalmic illumination device, comprising: an ophthalmic lens portion for interacting with a patient's eye; one or more light-emitting diodes (LEDs) structurally integrated with or forming a detachable module for the lens portion, positioned to illuminate an intraocular region; a miniature battery embedded within the device or detachable module, directly powering the one or more LEDs; and control circuitry configured to drive the one or more LEDs with a current exceeding a standard operating current specification of the LEDs.
Clause 2. A high-brightness, single-use ophthalmic illumination module, comprising: a housing configured to couple to an ophthalmic lens; at least one LED disposed within the housing; a battery disposed within the housing; and driver electronics configured to draw current from the battery at a rate exceeding the battery's specified maximum continuous discharge rate to power the at least one LED.
Clause 3. An ophthalmic illumination system optimized for disposability, comprising: a disposable illumination unit configured to couple to an ophthalmic interface; a battery integrated within the disposable illumination unit; and one or more LEDs integrated within the disposable illumination unit; wherein the system is configured to operate the one or more LEDs at a brightness level requiring a drive current that reduces an expected operational lifetime of the battery or the LEDs to between 5 minutes and 100 minutes.
Clause 4. A method for providing high-brightness illumination in a single-use ophthalmic device, comprising: providing a disposable ophthalmic illumination device comprising an integrated battery and one or more LEDs; activating the device; intentionally overdriving the one or more LEDs with a drive current greater than 20 mA to maximize brightness; and simultaneously intentionally overdriving the integrated battery to supply the drive current, wherein the overdriving reduces an operational duration of the device to a time sufficient for a single ophthalmic procedure.
Clause 5. A method for operating a self-contained ophthalmic illumination device, comprising: providing an illumination device comprising a lens interface, one or more LEDs, and an integrated Li-Ion battery; coupling the illumination device to an eye; and driving the one or more LEDs with a current between 25 mA and 100 mA supplied by the integrated Li-Ion battery.
Clause 6. The device, module, system, or method of any of Clauses 1-5, wherein the drive current is between 25 mA and 100 mA per LED.
Clause 7. The device, module, system, or method of any of Clauses 1-5, wherein the battery is a Lithium-Ion (Li-Ion) battery.
Clause 8. The device, module, system, or method of any of Clauses 1-5, wherein the battery has a specified maximum continuous discharge rate of less than 20 mA, and the driver electronics draw current exceeding 20 mA.
Clause 9. The device, module, system, or method of any of Clauses 1-5, wherein the operational duration or lifetime is between 5 minutes and 60 minutes.
Clause 10. The device, module, system, or method of any of Clauses 1-5, wherein the device, module, or unit is configured to be discarded after the single ophthalmic procedure or when the battery is depleted.
Clause 11. The device, module, system, or method of any of Clauses 1-5, wherein the miniature battery has a thickness between 1 mm and 5 mm and a diameter between 6 mm and 12 mm.
Clause 12. The device, module, system, or method of any of Clauses 1-5, wherein the battery is a LIR 1040 type battery.
Clause 13. The device, module, system, or method of any of Clauses 1-5, further comprising a user-operable control element on the device or module for activating the overdrive mode.
Clause 14. The device, module, system, or method of any of Clauses 1-5, wherein the device or module is cordless.
Clause 15. The device, module, system, or method of any of Clauses 1-5, wherein the control circuitry or driver electronics include a microcontroller configured to manage the drive current.
Clause 16. The device, module, system, or method of any of Clauses 1-5, further comprising one or more feedback indicators configured to convey status information including battery level.
Clause 17. The device, module, system, or method of any of Clauses 1-5, wherein the detachable module or housing is a cap containing the LEDs, battery, and control circuitry.
Clause 18. The device, module, system, or method of any of Clauses 1-5, wherein the battery is a single-use, non-rechargeable battery.
Clause 19. The device, module, system, or method of any of Clauses 1-5, wherein the battery is a rechargeable battery utilized for a single charge cycle.
Clause 20. The device, module, system, or method of any of Clauses 1-5, wherein the device or module is lightweight and designed not to significantly alter the maneuverability of an associated ophthalmic lens.
Clause 21. The device, module, system, or method of any of Clauses 1-5, wherein the ophthalmic lens portion or interface is a gonioscope.
Clause 22. The device, module, system, or method of any of Clauses 1-5, wherein the control circuitry includes circuitry configured to maintain stable light output despite battery discharge during the operational duration.
Clause 23. The device, module, system, or method of any of Clauses 1-5, wherein the device or module is designed for waterproof characteristics to withstand cleaning or sterilization processes prior to use.
Clause 24. The device, module, system, or method of any of Clauses 1-5, further comprising an external power port configured to bypass the battery and power the LEDs from an external source.
Clause 25. The device, module, system, or method of any of Clauses 1-5, wherein the control circuitry is configured to disable the device after a predetermined operational time has elapsed.
Clause 26. The method of any of Clauses 4-25, wherein the single ophthalmic procedure has a duration of less than 60 minutes.
Clause 27. The method of any of Clauses 4-26, further comprising discarding the disposable ophthalmic illumination device after the battery is depleted or the procedure is complete.
Clause 28. The method of any of Clauses 4-27, wherein the integrated battery is a Li-Ion battery.
Clause 29. The method of any of Clauses 4-28, wherein overdriving the battery comprises drawing current at a rate exceeding the battery's specified maximum continuous discharge rate.
Clause 30. The method of any of Clauses 5-29, wherein the integrated Li-Ion battery has a thickness of 1 mm to 5 mm and a diameter of 6 mm to 12 mm.
Clause 31. The device, module, system, or method of any of Clauses 1-5, comprising multiple LEDs, wherein the total current drawn from the battery exceeds 50 mA.
Clause 32. The device, module, system, or method of any of Clauses 1-5, wherein the control circuitry comprises simple resistors to drive the LEDs.
Clause 33. The device, module, system, or method of any of Clauses 1-5, further comprising integrated cameras powered by the miniature battery, wherein the battery is overdriven to supply current to both the LEDs and the cameras.
Clause 34. The method of any of Clauses 4-33, further comprising maneuvering the device on the eye without encumbrance from external cables.
Clause 35. The method of any of Clauses 4-34, wherein the high-brightness illumination enhances visualization for high-precision laser focus alignment.
Clause 36. The device, module, system, or method of any of Clauses 1-5, wherein the device is sterilized via gamma irradiation, electron beam, or EtO prior to packaging.
Clause 37. The device, module, system, or method of any of Clauses 1-5, wherein the LEDs are configured to emit white light.
Clause 38. The device, module, system, or method of any of Clauses 1-5, wherein the illumination provides standardized, pre-calibrated lighting intensity facilitated by the overdrive configuration.
Clause 39. The method of any of Clauses 4-38, wherein the method reduces surgeon workload by providing sufficient illumination without requiring adjustment of external light sources.
Clause 40. The device, module, system, or method of any of Clauses 1-5, wherein the device provides consistent illumination quality for the duration of the procedure despite the overdriven state.

### Clause Set 8: Office-Based Surgical Workflow and Interface

Clause 1. A surgical interface system for performing office-based ophthalmic mixed procedures, comprising: an ophthalmic contact lens body configured to visualize an intraocular structure; an illumination module mechanically coupled to the contact lens body, comprising an integrated power source and at least one light source oriented to illuminate the intraocular structure; and a surgical access port defined by a physical notch, gap, or open arc segment on the contact lens body, wherein the surgical access port is sized and positioned to provide mechanical clearance for a surgical instrument or drug delivery injector to enter the eye while the illumination module remains attached to the contact lens body.
Clause 2. The system of Clause 1, wherein the ophthalmic contact lens body is a gonioscopic lens and the surgical access port is positioned to allow a minimally invasive glaucoma surgery (MIGS) device or drug injector to access the anterior chamber angle.
Clause 3. The system of any of the preceding Clauses, further comprising at least one miniature camera integrated into the illumination module and configured to transmit live video of the illuminated intraocular structure to an external monitor, thereby providing a heads-up display for a user.
Clause 4. The system of Clause 3, wherein the miniature camera comprises an autofocus mechanism configured to maintain focus on the intraocular structure during manipulation of the contact lens body.
Clause 5. The system of any of the preceding Clauses, wherein the illumination module is a ring-shaped or partially ring-shaped cap that snaps onto an upper rim of the contact lens body and includes a central alignment aperture aligned with an optical pathway of the contact lens body.
Clause 6. The system of any of the preceding Clauses, wherein the surgical access port comprises an open region of the ring-shaped illumination module that leaves a portion of the contact lens body uncovered to permit direct instrument access while maintaining illumination of an intraocular target.
Clause 7. The system of any of the preceding Clauses, wherein the illumination module comprises a self-contained battery integrated into the module such that the system is cordless during operation.
Clause 8. The system of any of the preceding Clauses, wherein the surgical access port is positioned to allow an injector or MIGS device to traverse the anterior chamber from a temporal approach to access a nasal angle while avoiding collision with the contact lens body and illumination module.
Clause 9. The system of any of the preceding Clauses, further comprising status indicator lights on the illumination module configured to convey at least one of power status, battery level, or laser ready status.
Clause 10. The system of any of the preceding Clauses, further comprising at least one miniature camera integrated into the illumination module to provide live video for heads-up viewing during the mixed procedure.
Clause 11. A method for performing a mixed diagnostic and therapeutic ophthalmic procedure without a slit lamp, comprising: positioning a patient in a reclined orientation; applying a self-illuminated contact lens assembly to the patient's eye, the assembly comprising an integrated light source and battery; holding the self-illuminated contact lens assembly with a first hand to visualize a target tissue within the eye; and inserting a therapeutic device into the eye with a second hand through a surgical access notch in the lens assembly to deliver a pharmacological agent or implant to the target tissue.
Clause 12. The method of Clause 11, wherein visualizing the target tissue comprises viewing a video feed from a camera integrated into the contact lens assembly on an external monitor.
Clause 13. The method of any of Clauses 11-12, wherein visualizing the target tissue comprises viewing a video feed from a camera integrated into the contact lens assembly in a virtual headset or augmented reality glasses.
Clause 14. The method of any of Clauses 11-13, wherein positioning the patient in a reclined orientation comprises placing the patient in a supine or semi reclined position at approximately 45 degrees.
Clause 15. The method of any of Clauses 11-14, wherein the self-illuminated contact lens assembly includes status indicators and a user operable control to adjust illumination intensity or wavelength prior to inserting the therapeutic device.
Clause 16. The method of any of Clauses 11-15, wherein the therapeutic device is advanced from a temporal entry site to deliver a sustained release implant to the nasal anterior chamber angle through the surgical access notch while the illumination remains active.
Clause 17. The method of any of Clauses 11-16, wherein visualizing the target tissue further comprises utilizing a heads up display receiving a live video feed from a miniature camera integrated into the lens assembly.
Clause 18. The method of any of Clauses 11-17, wherein the illumination is provided by a battery powered, self-contained module attached to the lens assembly, eliminating the need for a slit lamp or external light cables during the procedure.

### Clause Set 9: Automated Diagnostics and Laser Metrology

Clause 1. An automated ophthalmic diagnostic system, comprising: a multi-mirror contact lens comprising a plurality of mirrors arranged to visualize different quadrants of an anterior chamber angle; an attachment module coupled to the contact lens; a plurality of miniature cameras integrated into the attachment module, wherein each camera is aligned with a respective mirror of the plurality of mirrors; and a processor configured to simultaneously capture images from the plurality of miniature cameras to generate a comprehensive 360-degree assessment of the anterior chamber angle.
Clause 2. The system of Clause 1, wherein the multi mirror contact lens comprises a four mirror gonioscopy lens and the plurality of miniature cameras comprises four cameras each aligned with a corresponding mirror.
Clause 3. The system of any of the preceding Clauses, wherein the attachment module further includes illumination sources oriented to direct light toward each mirror's field of view to standardize lighting across all quadrants during simultaneous capture.
Clause 4. The system of any of the preceding Clauses, wherein the processor is configured to analyze the simultaneous images to automatically determine a pigmentation grade of the trabecular meshwork.
Clause 5. The system of any of the preceding Clauses, wherein the plurality of miniature cameras are configured to transmit image data wirelessly from the attachment module to an external processor.
Clause 6. The system of any of the preceding Clauses, wherein the attachment module is configured as a detachable ring that aligns concentrically with an optical axis of the contact lens to maintain camera to mirror registration.
Clause 7. The system of any of the preceding Clauses, wherein the processor is configured to analyze the captured images to automatically determine a pigmentation grade of the trabecular meshwork.
Clause 8. An ophthalmic diagnostic device for quantifying anterior angle openness, comprising: a contact lens interface; a structured light emitter integrated into the device, configured to project a beam of light having a fixed, known geometry towards the anterior chamber angle of an eye; and an imaging sensor configured to capture the interaction of the projected beam with an iris and a corneoscleral shell of the eye; wherein the device is configured to determine an angle openness metric by detecting optical clipping of the projected beam by the iris.
Clause 9. The device of Clause 8, wherein the structured light emitter comprises a red laser diode configured to project a fan beam having a height of approximately 200 to 500 micrometers.
Clause 10. The device of any of Clauses 8-9, wherein the imaging sensor is an integrated miniature camera mounted on the device to capture the incidence of the structured beam on the trabecular meshwork and iris.
Clause 11. The device of any of Clauses 8-10, wherein the device is configured to compute the angle openness metric as a ratio between a visible portion of the projected beam on the angle structures and a portion optically clipped by the iris.
Clause 12. The device of any of Clauses 8-11, wherein the structured light emitter and the imaging sensor are mechanically coupled to the contact lens interface so that their relative geometry remains fixed during measurement.
Clause 13. The device of any of Clauses 8-12, wherein the device includes control electronics configured to standardize illumination intensity and acquisition timing during the angle openness measurement.
Clause 14. An ophthalmic imaging apparatus, comprising: an annular housing configured for attachment to a gonioscopic lens; a rotating carriage disposed within the annular housing; and at least one camera mounted on the rotating carriage; wherein the rotating carriage is motorized to rotate the at least one camera 360 degrees around an optical axis of the gonioscopic lens to capture a panoramic scan of the eye.
Clause 15. The apparatus of Clause 14, wherein the annular housing defines a central aperture aligned with an optical axis of the gonioscopic lens, and the rotating carriage maintains the at least one camera at a fixed radial distance from the axis during rotation.
Clause 16. The apparatus of any of Clauses 14-15, wherein the rotating carriage further comprises an illumination source co-rotating with the at least one camera to provide uniform lighting during the panoramic scan.
Clause 17. The apparatus of any of Clauses 14-16, wherein the motorized rotating carriage is configured to capture a complete 360-degree image sequence for automated panoramic reconstruction of the anterior chamber angle.
Clause 18. The apparatus of any of Clauses 14-17, wherein image data from the at least one camera is transmitted via a wired or wireless data interface to an external processor for real-time display.
Clause 19. The apparatus of any of Clauses 14-18, wherein the annular housing is configured as a detachable ring that mounts onto an upper rim of the gonioscopic lens.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 10% percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. An ophthalmic illumination apparatus, comprising:
an ophthalmic lens body configured for direct contact with or close proximity to a patient's eye to facilitate viewing or treatment of an intraocular structure; and
at least one miniature illumination source coupled to the ophthalmic lens body and positioned to emit light towards the intraocular structure, wherein the emitted light from the at least one miniature illumination source moves in unison with the ophthalmic lens body as the ophthalmic lens body is moved or reoriented relative to the eye, thereby maintaining automatically aligned illumination of a region of interest within the eye.

2. The apparatus of claim 1, wherein the ophthalmic lens body is a gonioscopic lens comprising at least one mirror, and wherein the at least one miniature illumination source is positioned and oriented to illuminate a region of the anterior chamber angle viewable via the at least one mirror.

3. The apparatus of claim 1, wherein the at least one miniature illumination source is embedded within the material of the ophthalmic lens body adjacent to an optical element of the lens body.

4. The apparatus of claim 1, further comprising at least one miniature camera coupled to the ophthalmic lens body, the camera positioned to capture images of the illuminated intraocular structure.

5. The apparatus of claim 1, further comprising a controller configured to adjust at least one of an intensity, a wavelength, a polarization state, or a timing of the emitted light.

6. The apparatus of claim 1, wherein the at least one miniature illumination source is configured to provide illumination of selectable different colors, including at least one of white, green, blue, red, cyan, magenta, or infrared light.

7. The apparatus of claim 1, further comprising a polarizer associated with the at least one miniature illumination source to emit polarized light.

8. The apparatus of claim 1, further comprising an alignment aperture integrated with the ophthalmic lens body or an attachment thereto, configured to facilitate alignment of a treatment beam or viewing path.

9. The apparatus of claim 1, wherein the ophthalmic lens body is a direct contact lens for vitreoretinal viewing, and the at least one miniature illumination source is positioned to illuminate the posterior segment of the eye.

10. The apparatus of claim 1, wherein the ophthalmic lens body is a multi-mirror lens, and the apparatus comprises multiple illumination sources, wherein at least one illumination source is dedicated to each mirror.

11. The apparatus of claim 1, further comprising a central illumination source positioned to emit light coaxially through a pupil of the eye.

12. The apparatus of claim 1, further comprising status indicator lights integrated with the ophthalmic lens body or an attachment thereto.

13. The apparatus of claim 1, further comprising a user-operable control element on the ophthalmic lens body or an attachment thereto for controlling the illumination source.

14. The apparatus of claim 1, further comprising a power source integrated within the ophthalmic lens body.

15. The apparatus of claim 14, wherein the at least one miniature illumination source is configured to be overdriven with a current greater than 20 mA supplied by a self-contained power source.

16. An ophthalmic illumination system, comprising:
an ophthalmic patient interface device adapted to be placed on or coupled to an eye;
an illumination module mechanically attachable to and detachable from the ophthalmic patient interface device, the illumination module comprising a plurality of miniature light-emitting elements arranged to direct light through the ophthalmic patient interface device or alongside the ophthalmic patient interface device towards an intraocular target region when the illumination module is attached; and
a power source configured to energize the plurality of miniature light-emitting elements.

17. The system of claim 16, wherein the illumination module is a ring-shaped or partially ring-shaped cap configured to snap onto or encircle an upper portion of the ophthalmic patient interface device.

18. The system of claim 16, wherein the illumination module defines a central aperture aligned with an optical pathway of the ophthalmic patient interface device.

19. The system of claim 16, further comprising at least one miniature camera integrated into the illumination module, positioned to capture images of the illuminated intraocular target region.

20. The system of claim 19, wherein the at least one miniature camera comprises two cameras arranged to provide stereoscopic imaging.

21. The system of claim 16, further comprising control electronics integrated within the illumination module for managing power and illumination parameters.

22. A method for illuminating an intraocular structure, the method comprising:
placing an ophthalmic lens assembly onto a patient's eye, the ophthalmic lens assembly comprising an ophthalmic lens and at least one integrated or detachably coupled miniature illumination source;
activating the at least one miniature illumination source to direct light towards a target intraocular structure viewable through or facilitated by the ophthalmic lens; and
maneuvering the ophthalmic lens assembly relative to the eye, wherein the illumination from the at least one miniature illumination source automatically follows the maneuvering to maintain illumination of the target intraocular structure without manual adjustment of an external light source.
